(19) United States
(12) Patent Application Publication
(10) Pub. No.: US 2024/0358280 A1
(43) Pub. Date: Oct. 31, 2024

(54) INSERTION NEEDLE STRUCTURE AND INSERTER

(71) Applicant: BIONIME CORPORATION, Taichung City (TW)

(72) Inventors: Li-Kang HUANG, Taichung City (TW); Chieh-Hsing CHEN, Taichung City (TW); Tsung-Da LI, Taichung City (TW)

(21) Appl. No.: 18/765,375

(22) Filed: Jul. 8, 2024

Related U.S. Application Data
(63) Continuation-in-part of application No. 17/450,834, filed on Oct. 14, 2021, now Pat. No. 12,053,277.
(60) Provisional application No. 63/091,318, filed on Oct. 14, 2020.

(51) Int. Cl. A61B 5/145; A61B 17/34

(52) U.S. Cl. CPC A61B 5/14503; A61B 17/3417; A61B 17/3468; A61B 2017/3454; A61B 2560/063

(57) ABSTRACT

An insertion needle structure includes a needle sharp and a needle body. The needle body is integrally connected to the needle sharp and has a receiving space for receiving the biosensor. The needle body includes a base wall, two side walls, two slope sections and two curved connecting sections. The side walls are located at two sides of the base wall, respectively, the side walls are at least partially nonparallel, and each of the side walls is at least partially flat. Each of the slope sections is connected between each of the side walls and the needle sharp, and each of the slope sections is curved. Each of the curved connecting sections is connected between each of the side walls and the base wall and between each of the slope sections and the base wall. The needle sharp extends from the base wall and the curved connecting sections.

### RELATED APPLICATIONS

This application is a Continuation-in-part of U.S. application Ser. No. 17/450,834, filed on Oct. 14, 2021, which claims priority to U.S. Provisional Application Ser. No. 63/091,318, filed Oct. 14, 2020, the contents of which are incorporated herein by reference.

### BACKGROUND

### Technical Field

The present disclosure relates to an insertion needle and an inserter. Particularly, the present disclosure relates to an insertion needle and an inserter applied for inserting a biosensor.

### Description of Related Art

Glucose monitor inside the body is very important for diabetes patients. In addition, the specific physiological parameters, such as blood fatty and the content of cholesterol, of the patients with the chronic illness have to be daily monitored for tracking the illness condition, thereby assisting the latter treatment. Generally, such physiological parameters are obtained by extracting the body fluid of the patient for further analysis, and, for example, a conventional glucose meter employs a needle to pierce the skin surface of the human body to extract the blood for analyzing the value of the glucose.

However, in order to improve the accuracy and immediacy of the monitor, a biosensor which can be implanted underneath the skin surface of the human body is developed. Through the biosensor, real-time physiological parameters can be obtained. The physiological parameters can be sent to the cloud or the back-end monitoring system in association with the signal processer, and numerous and immediate analyzed data can be provided, which prevents the discomfort and the risk of infection caused by invasive extractions of the body fluid.

The biosensor can be implanted underneath the skin surface of the human body by an inserter. The inserter can include an insertion needle, and the biosensor can be received in the insertion needle. By using the insertion needle to pierce the skin surface of the human body to form a small aperture, the biosensor can enter the aperture so as to be implanted underneath the skin surface of the human body. If the aperture is too large or non-smooth, the aperture, i.e., the wound, cannot heal quickly. Hence, how to improve the structure of the insertion needle to lower the burrs and increase the insertion smoothness for increasing the flatness of the aperture formed on the skin surface of the human body or the organism becomes a pursued target for practitioners.

### SUMMARY

According to one aspect of the present disclosure, an insertion needle structure which is formed by bending a flat blank and is configured for receiving and allowing a biosensor to be partially implanted underneath a skin surface of an organism includes a needle sharp and a needle body. The needle body is integrally connected to the needle sharp and has a receiving space for receiving the biosensor. The needle body includes a base wall, two side walls, two slope sections and two curved connecting sections. The two side walls are located at two sides of the base wall, respectively, the two side walls are at least partially nonparallel, and each of the two side walls is at least partially flat. The two slope sections are located at the two sides of the base wall, respectively, each of the slope sections is connected between each of the side walls and the needle sharp, and each of the slope sections is curved. The two curved connecting sections are located at the two sides of the base wall, and each of the curved connecting sections is connected between each of the side walls and the base wall and between each of the slope sections and the base wall. The needle sharp extends from the base wall and the curved connecting sections.

According to still yet another aspect of the present disclosure, an inserter includes a cover having a main space, an inserting module disposed within the main space of the cover and including the abovementioned insertion needle structure, and a removing module including a base and the biosensor. The base is detachably limited within the inserting module. The biosensor is detachably assembled with the base and at least a part thereof is received in the receiving space of the insertion needle structure. When the cover is pressed downward, the inserting module is driven to allow the insertion needle structure to move downward so as to carry the biosensor to implant underneath the skin surface of the organism for conducting a measurement of a physiological signal inside the organism.

### BRIEF DESCRIPTION OF THE DRAWINGS

The disclosure can be more fully understood by reading the following detailed description of the embodiments, with reference made to the accompanying drawings as follows:

FIG. 1 shows a three-dimensional schematic view of an insertion needle structure according to a first embodiment of the present disclosure.

FIG. 2 shows a front view of the insertion needle structure of the first embodiment of FIG. 1.

FIG. 3 shows a side view of the insertion needle structure of the first embodiment of FIG. 1.

FIG. 4 shows a top view of the insertion needle structure of the first embodiment of FIG. 1.

FIG. 5 shows a top view of a flat blank used for being bended and forming the insertion needle structure of the first embodiment of FIG. 1.

FIG. 6 shows one three-dimensional schematic view of an insertion needle structure according to a second embodiment of the present disclosure.

FIG. 7 shows a front view of the insertion needle structure of the second embodiment of FIG. 6.

FIG. 8 shows another three-dimensional schematic view of the insertion needle structure of the second embodiment of FIG. 6.

FIG. 9 shows one three-dimensional schematic view of an insertion needle structure according to a third embodiment of the present disclosure.

FIG. 10 shows another three-dimensional schematic view of the insertion needle structure of the third embodiment of FIG. 9.

FIG. 11 shows a front view of the insertion needle structure of the third embodiment of FIG. 9.

FIG. 12 shows a top view of the insertion needle structure of the third embodiment of FIG. 9.

FIG. 13 shows a side view of the insertion needle structure of the third embodiment of FIG. 9.

FIG. 14 shows a three-dimensional schematic view of an insertion needle structure according to a fourth embodiment of the present disclosure.

FIG. 15 shows a front view of the insertion needle structure of the fourth embodiment of FIG. 14.

FIG. 16 shows a side view of the insertion needle structure of the fourth embodiment of FIG. 14.

FIG. 17 shows a top view of a flat blank used for being bended and forming the insertion needle structure of the fourth embodiment of FIG. 14.

FIG. 18 shows a front section view of an insertion needle structure according to a fifth embodiment of the present disclosure.

FIG. 19 shows a top view of a flat blank used for being bended and forming the insertion needle structure of the fifth embodiment of FIG. 18.

FIG. 20 shows a three-dimensional schematic view of an insertion needle structure according to a sixth embodiment of the present disclosure.

FIG. 21 shows a three-dimensional schematic view of an insertion needle structure according to a seventh embodiment of the present disclosure.

FIG. 22 shows a side view of the insertion needle structure of the seventh embodiment of FIG. 21.

FIG. 23 shows a three-dimensional schematic view of an insertion needle structure according to an eighth embodiment of the present disclosure.

FIG. 24 shows an exploded three-dimensional schematic view of an inserter according to a ninth embodiment of the present disclosure.

FIG. 25 shows a partial section view of the inserter of the ninth embodiment of FIG. 24.

FIG. 26 shows a three-dimensional schematic view of an insertion needle structure according to a tenth embodiment of the present disclosure.

FIG. 27 shows a cross-sectional view of the insertion needle structure of the tenth embodiment of FIG. 26 taken along line 27-27.

FIG. 28 shows a cross-sectional view of the insertion needle structure of the tenth embodiment of FIG. 26 taken along line 28-28.

FIG. 29 shows a top view of the insertion needle structure of the tenth embodiment of FIG. 26.

FIG. 30 shows a side view of the insertion needle structure of the tenth embodiment of FIG. 26.

FIG. 31 shows a three-dimensional schematic view of an insertion needle structure according to an eleventh embodiment of the present disclosure.

FIG. 32 shows a cross-sectional view of the insertion needle structure of the eleventh embodiment of FIG. 31 taken along line 32-32.

FIG. 33 shows a cross-sectional view of the insertion needle structure of the eleventh embodiment of FIG. 31 taken along line 33-33.

FIG. 34 shows a top view of the insertion needle structure of the eleventh embodiment of FIG. 31.

FIG. 35 shows a side view of the insertion needle structure of the eleventh embodiment of FIG. 31.

FIG. 36 shows a three-dimensional schematic view of an insertion needle structure according to a twelfth embodiment of the present disclosure.

FIG. 37 shows a cross-sectional view of the insertion needle structure of the twelfth embodiment of FIG. 36 taken along line 37-37.

FIG. 38 shows a top view of the insertion needle structure of the twelfth embodiment of FIG. 36.

FIG. 39 shows a side view of the insertion needle structure of the twelfth embodiment of FIG. 36.

FIG. 40 shows a three-dimensional schematic view of an insertion needle structure according to a thirteenth embodiment of the present disclosure.

FIG. 41 shows a cross-sectional view of the insertion needle structure of the thirteenth embodiment of FIG. 40 taken along line 41-41.

FIG. 42 shows a top view of the insertion needle structure of the thirteenth embodiment of FIG. 40.

FIG. 43 shows a side view of the insertion needle structure of the thirteenth embodiment of FIG. 40.

FIG. 44 shows a cross-sectional view of an insertion needle structure according to a fourteenth embodiment of the present disclosure.

### DETAILED DESCRIPTION

It will be understood that when an element (or mechanism or module) is referred to as being “disposed on”, “connected to” or “coupled to” another element, it can be directly disposed on, connected or coupled to another element, or it can be indirectly disposed on, connected or coupled to another element, that is, intervening elements may be present. In contrast, when an element is referred to as being “directly disposed on”, “directly connected to” or “directly coupled to” another element, there are no intervening elements present.

In addition, the terms first, second, third, etc. are used herein to describe various elements or components, these elements or components should not be limited by these terms. Consequently, a first element or component discussed below could be termed a second element or component.

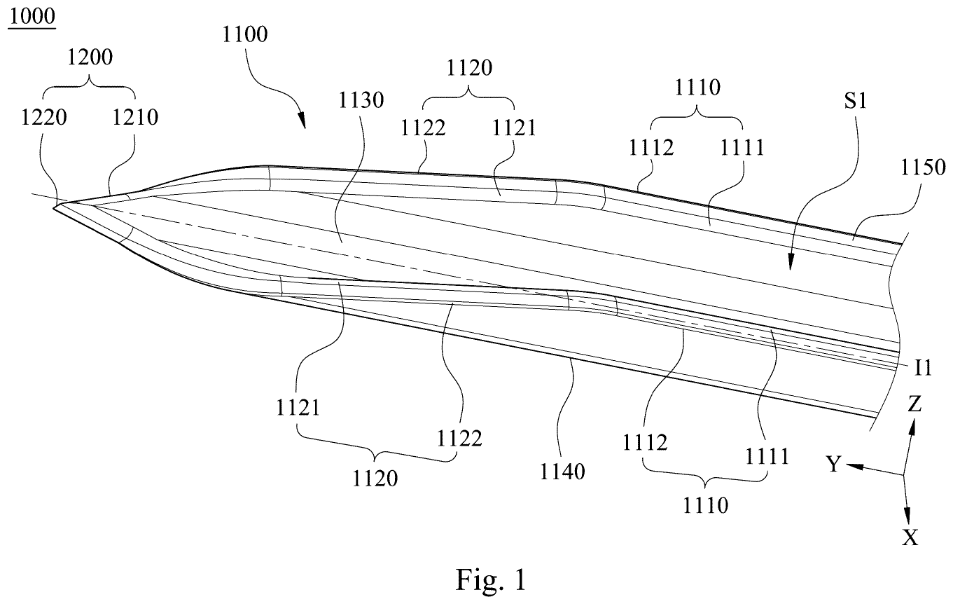

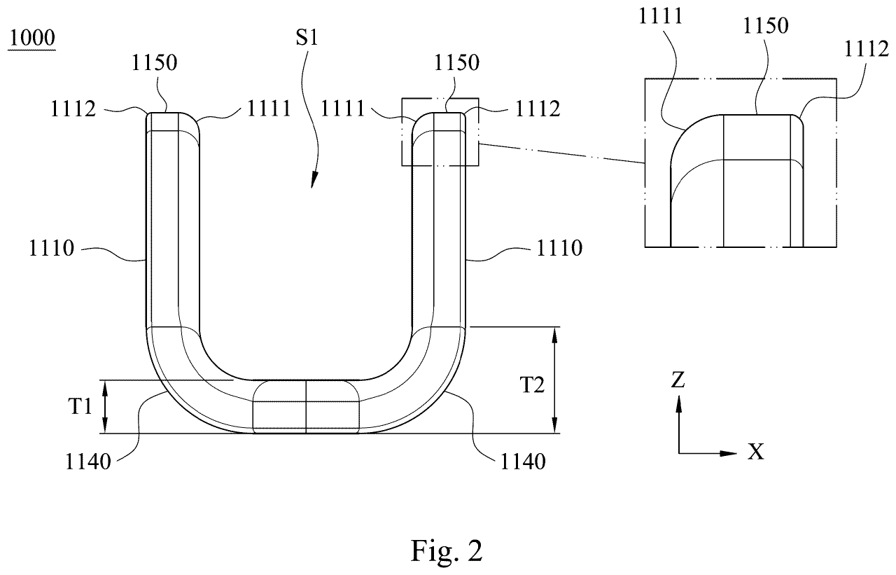

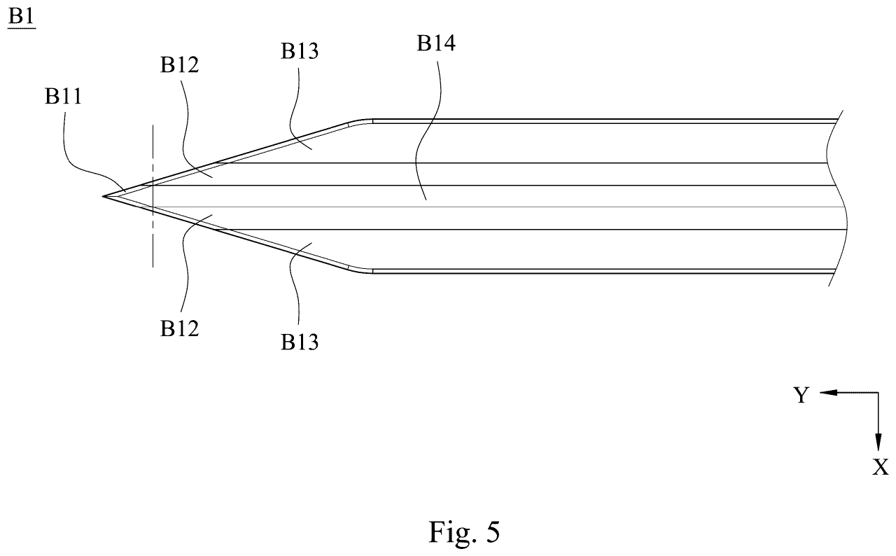

FIG. 1 shows a three-dimensional schematic view of an insertion needle structure 1000 according to a first embodiment of the present disclosure. FIG. 2 shows a front view of the insertion needle structure 1000 of the first embodiment of FIG. 1. Please refer to FIGS. 1 and 2, the insertion needle structure 1000 which is formed by bending a flat blank B1 (shown in FIG. 5) and is configured for receiving and allowing a biosensor (not shown in the first embodiment) to be partially implanted underneath a skin surface (not shown) of an organism includes a needle sharp 1200 and a needle body 1100. The needle body 1100 is integrally connected to the needle sharp 1200 and includes a base wall 1130, two side walls 1110, and two slope sections 1120. A receiving space S1 for receiving the biosensor is defined by the two side walls 1110, the two slope sections 1120, and the base wall 1130. The two side walls 1110 are located at two sides of the base wall 1130, respectively. Each of the side walls 1110 has a first inner edge 1111 and a first outer edge 1112. The first inner edge 1111 is near the receiving space S1, and the first outer edge 1112 faces away from the receiving space S1. The two slope sections 1120 are located at the two sides of the base wall 1130, respectively. Each of the slope sections 1120 is connected between each of the side walls 1110 and the needle sharp 1200. Each of the slope sections 1120 has a second inner edge 1121 connected to the first inner edge 1111, and a second outer edge 1122 is connected to the first outer edge 1112. Each of the first inner edges 1111, each of the second inner edges 1121, each of the first outer edges 1112 and each of the second outer edges 1122 are curved. R11 represents a radius of each of the first inner edges 1111, R12 represents a radius of each of the first outer edges 1112, and a condition of R11>R12 is satisfied.

Therefore, because each of the first inner edges 1111, each of the second inner edges 1121, each of the first outer edges 1112 and each of the second outer edges 1122 are curved, the insertion needle structure 1000 is favorable for smoothly piercing the skin surface of the organism, which can increase the flatness of the aperture formed on the skin surface of the organism. Moreover, through the condition of R11>R12, damage of the biosensor inside the receiving space S1 can be avoided. The details of the insertion needle structure 1000 will be described hereinafter.

The insertion needle structure 1000 is a three-dimensional structure. Without considering the thickness, the base wall 1130 is located on a plane formed by a length direction Y and a width direction X of the insertion needle structure 1000, and the side walls 1110 and the slope sections 1120 are located on the plane formed by the length direction Y and a height direction Z of the insertion needle structure 1000. One of the side walls 1110 and one of the slope sections 1120 are located on one side of the central axis I1 of the insertion needle structure 1000, and the other one of the side walls 1110 and the other one of the slope sections 1120 are located on the other side of the central axis I1 of the insertion needle structure 1000. The two side walls 1110 are aligned symmetrically, and the two slope sections 1120 are aligned symmetrically.

The needle body 1100 can further include two curved connecting sections 1140, and each of the curved connecting sections 1140 is connected between each of the side walls 1110 and the base wall 1130 and between each of the slope sections 1120 and the base wall 1130. In other words, the base wall 1130 located on the plane formed by the length direction Y and the width direction X can be smoothly connected to the side walls 1110 and the slope sections 1120 on the plane formed by the length direction Y and the height direction Z so as to form a cross-section being U-shaped. Furthermore, each of the curved connecting sections 1140 has a height thereof represented by T2 along the height direction Z of the insertion needle structure 1000, the flat blank B1 has a thickness represented by T1 which is identical to the thickness of the base wall 1130 and is marked on FIG. 2, and a condition of T2/T1≥1.5 is satisfied. When the condition is satisfied, the anti-bending capability of the needle sharp 1200 can be increased to enhance the piercing capability, and the piercing force can be decreased to lower the piercing pain.

The needle body 1100 can further include a connecting surface 1150 which is parallel to the width direction X of the insertion needle structure 1000 and connected between each of the first inner edges 1111 and each of the first outer edges 1112. In other words, the inner surface and the outer surface of the side wall 1110 can be vertical. The radius angle of the first inner edge 1111 and the radius angle of the first outer edge 1112 are both 90 degrees, but the radius of the first inner edge 1111 and the radius of the first outer edge 1112 are different. As shown in the enlarged schematic view in FIG. 2, the first inner edge 1111 and the first outer edge 1112 are connected to each other via the connecting surface 1150. The second inner edge 1121 and the second outer edge 1122 can also be connected via the connecting surface 1150. The side wall 1110 can have a substantially uniform height which is defined by the distance from the intersection between the curved connecting section 1140 and the side wall 1110 to the connecting surface 1150 along the height direction Z. The height of the starting position of the slope section 1120 is substantially equal to zero, the height of the slope section 1120 is incrementally increased along the length direction Y, and the height of the stop position of the slope section 1120 is substantially equal to the height of the side wall 1110, thereby allowing the slope section 1120 to be smoothly connected to the side wall 1110. In the first embodiment, in addition to the starting position and the stop position, a slope of the height of the slope section 1120 is constant.

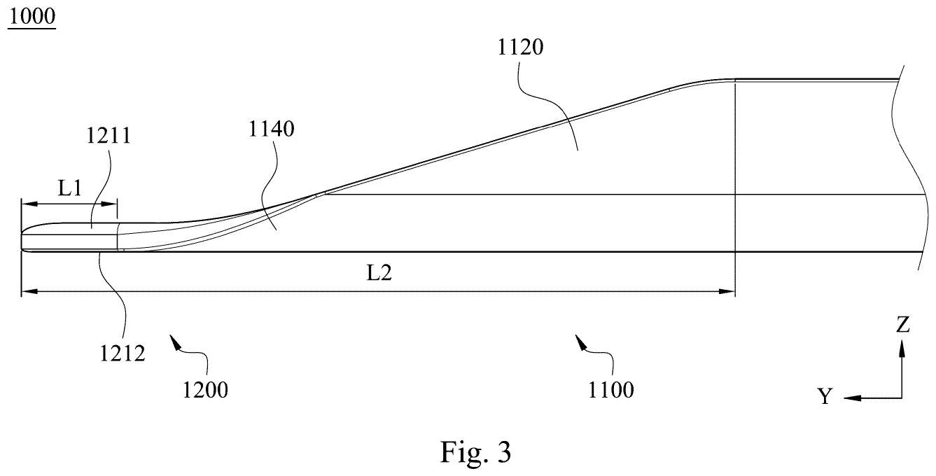

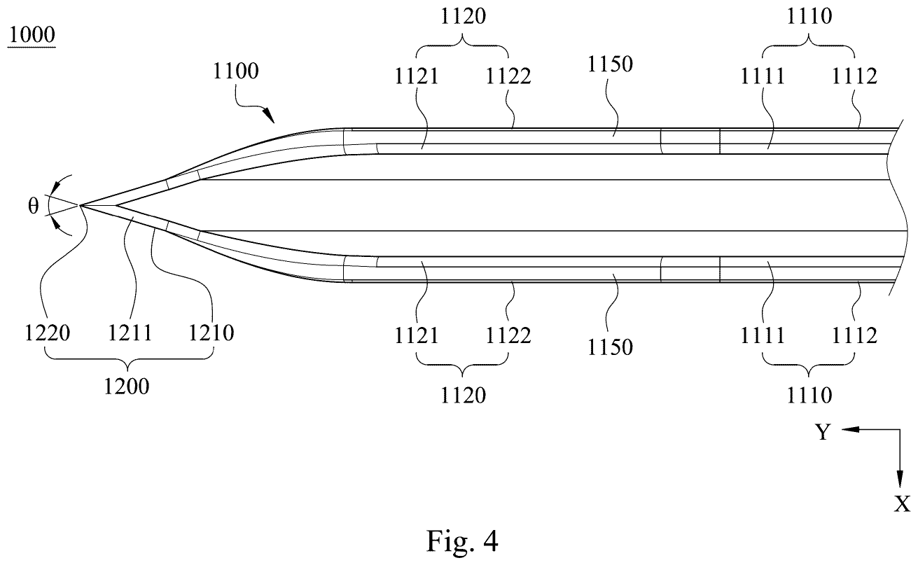

FIG. 3 shows a side view of the insertion needle structure 1000 of the first embodiment of FIG. 1. FIG. 4 shows a top view of the insertion needle structure 1000 of the first embodiment of FIG. 1. Please refer to FIGS. 3 and 4 with reference to FIGS. 1 and 2. The needle sharp 1200 can include two slants 1210 connected to the two slope sections 1120, respectively, and the two slants 1210 intersect at a needle tip 1220 with an angle θ. Each of the slants 1210 includes a needle sharp top edge 1211 being curved and connected to one of the two second inner edges 1121, and a needle sharp bottom edge 1212 being curved and connected to one of the two second outer edges 1122. R31 represents a radius of each of the needle sharp top edges 1211, R32 represents a radius of each of the needle sharp bottom edges 1212, and a condition of R31>R32 is satisfied. Moreover, the angle θ is within a range from 20 degrees to 40 degrees.

To be more specific, the needle sharp 1200 is substantially triangle-shaped, and, without considering the thickness, the needle sharp 1200 is located at the plane formed by the length direction Y and the width direction X. Each of the slants 1210 is indirectly connected to the slope section 1120 via the curved connecting section 1140, and the needle tip 1220 is located at the central axis I1. Please be noted that, the curved connecting section 1140 is smoothly connected to the side wall 1110 and the slope section 1120, and therefore the height of the curved connecting section 1140 in the height direction Z is incrementally decreased toward the slant 1210 along the length direction Y. Each of the curved connecting sections 1140 can further include a third inner edge (not labeled) and a third outer edge (not labeled), each of the needle sharp top edges 1211 is indirectly connected to the second inner edge 1121 via the third inner edge, and each of the needle sharp bottom edges 1212 is indirectly connected to the second outer edge 1122 via the third outer edge.

Moreover, L1 represents a needle sharp length defined by a distance along the length direction Y between the needle tip 1220 and a stop position of each of the slants 1210, L2 represents an expanding length defined by a distance along the length direction Y between the needle tip 1220 and a stop position of each of the slope sections 1120, and a condition of L1/L2≤15% is satisfied. The stop position of each of the slants 1210 is defined as the intersection between the slant 1210 and the curved connecting section 1140. The stop position of the slope section 1120 is defined as the intersection between the slope section 1120 and the side wall 1110. As the condition of L1/L2≤15% is satisfied, particularly L1/L2≤8%, the smoothness for expanding the aperture formed by insertion of the needle sharp 1200 into the skin surface of the organism can be increased, thereby favorable for implanting the biosensor.

FIG. 5 shows a top view of a flat blank B1 used for being bended and forming the insertion needle structure 1000 of the first embodiment of FIG. 1. Please refer to FIG. 5 with reference to FIGS. 1 to 4, the flat blank B1 can be made of a metal board, and the flat blank B1 can be bended to form the insertion needle structure 1000. Consequently, the thickness T1 of the flat blank B1 is identical to the thickness of the base wall 1130, and is identical to the thickness of each of the side wall 1110, the slope sections 1120 and the curved connecting sections 1140. Moreover, through the configuration that the bending radius of the flat blank B1 is equal to the thickness T1, the curved connecting section 1140 with the height T2 can be formed.

The flat blank B1 can be formed by a stamping process, especially a cutting process. During the manufacture for forming the flat blank B1, a portion which is defined to form the needle sharp 1200, i.e., the needle sharp portion B11, is processed by the stamping mold, and then the area to be cut is continuously processed by the stamping mold for further process such as shaving to define the contour and to enhance the sharpness of the needle sharp 1200. As a result, a burr height formed as the flat blank B1 stamped from the sheet is smaller than or equal to 0.02 mm. A finishing surface can be formed as the flat blank B1 stamped from the sheet, the finishing surface has a depth represented by T3 (not shown), and the depth T3 of the finishing surface and the thickness T1 of the flat blank B1 satisfy a condition of T3/T1≥50%, particularly T3/T1≥70%, more particularly T3/T1≥90%. Through the manufacture process, the contour of the flat blank B1 can be a continuous and uniform cutting face, and the process for modifying the surface and reducing the burrs can be omitted.

In the first embodiment, the radius of each of the first inner edges 1111 is represented by R11, the radius of each of the second inner edges 1121 is represented by R21, and the radius of each of the needle sharp top edges 1211 is represented by R31. The radius of each of the first outer edges 1112 is represented by R12, the radius of each of the second outer edges 1122 is represented by R22, and the radius of each of the needle sharp bottom edges 1212 is represented by R32. Conditions of R11=R21=R31 and R12=R22=R32 are satisfied. Moreover, each of the first outer edges 1112, each of the second outer edges 1122 and each of the needle sharp bottom edges 1212 are formed as the flat blank B1 stamped and elastic deformed from the sheet. Precisely, during stamping, the area to be cut will first be elastic deformed, then be plastically deformed, and finally be torn off. Therefore, the flat blank B1 which is completely separated from the sheet can be formed. As viewing from the side, a rollover zone and the rest, which is represented by a shear zone, caused by stamping the flat blank B1 can be formed. The rollover zone is curved owing to the elastic deformation, and can be used as the first outer edges 1112, the second outer edges 1122 and the needle sharp bottom edges 1212 without further processes. Consequently, the condition of 20%≤R11/T1≤50% can be satisfied. The shear zone is caused by plastic deformation, and the finishing surface is originally about 30% to 50% of the thickness T1 of the flat blank B1. The present disclosure can use the stamping mold and the process such as the shaving to increase the depth of the finishing surface to about more than 50% of the thickness T1 or about more than 70% of the thickness T1. Additionally, at least a part of the remained burrs can be rounded to form the first inner edges 1111, the second inner edges 1121 and the needle sharp top edges 1211, and a condition of 3≤R11/R12≤10 can be satisfied. As a result, the flat area of the cutting face of the flat blank B1 can be decreased and the remained small burrs can be removed. The friction between the insertion needle structure 1000 bended therefrom and the skin surface of the organism can be lowered during the inserting process.

The flat blank B1 can include a needle sharp portion B11, a base wall portion B14, two radius angle portions B12 and two wing portions B13. The needle sharp portion B11 is substantially triangle-shaped, and the base wall portion B14 can be strip-shaped and can be integrally connected to the needle sharp portion B11. A width of the base wall portion B14 is equal to the maximum width of the needle sharp portion B11. Each of the radius angle portions B12 is integrally connected to the base wall portion B14 and has an inclined line extending from the needle sharp portion B11. Each of the wing portions B13 is integrally connected to the radius angle portion B12 and has an inclined line extending from the radius angle portion B12, which has the same slope of the inclined line extending from the needle sharp portion B11, and a straight line connected to the inclined line. After the flat blank B1 is bended, the needle sharp portion B11 forms the needle sharp 1200, the radius angle portion B12 forms the curved connecting section 1140, and the wing portion B13 forms the side wall 1110 and the slope section 1120, thereby completing the insertion needle structure 1000.

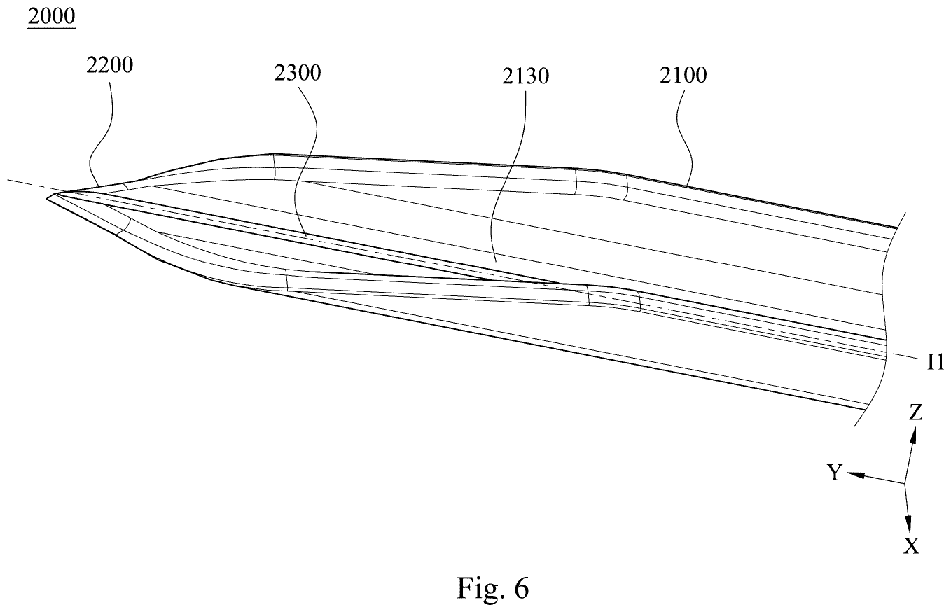

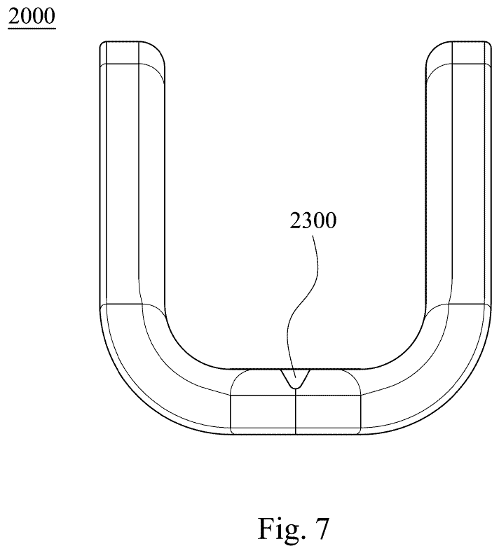

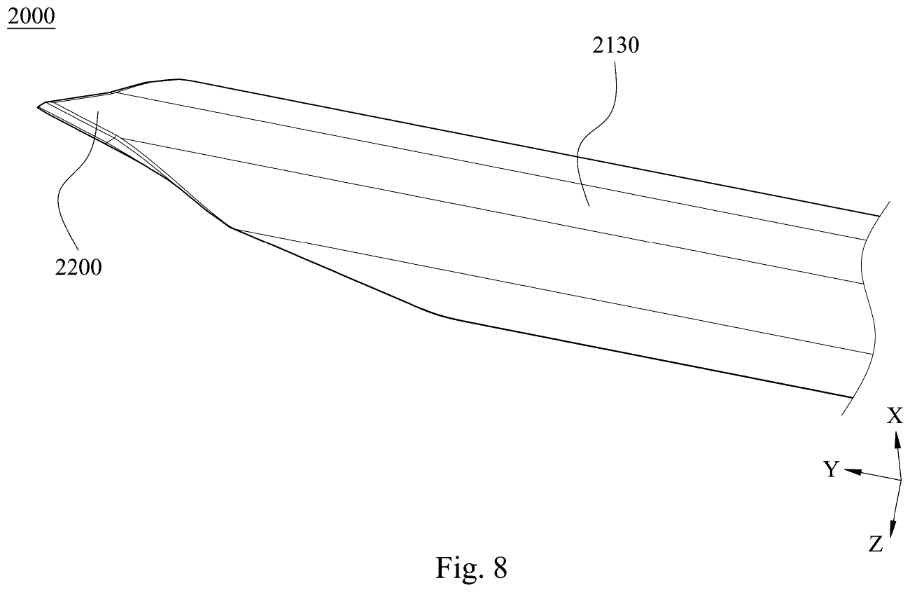

FIG. 6 shows one three-dimensional schematic view of an insertion needle structure 2000 according to a second embodiment of the present disclosure. FIG. 7 shows a front view of the insertion needle structure 2000 of the second embodiment of FIG. 6. FIG. 8 shows another three-dimensional schematic view of the insertion needle structure 2000 of the second embodiment of FIG. 6. The insertion needle structure 2000 is similar to the insertion needle structure 1000 of the first embodiment and includes a needle sharp 2200 and a needle body 2100. The difference is that the insertion needle structure 2000 can further include a reinforcing portion (not labeled). The reinforcing portion is disposed at at least one of the needle sharp 2200 and the needle body 2100 along the length direction Y. Particularly, a reinforcing area is defined as the needle sharp 2200 and a part of the needle body 2100 adjacent to the needle sharp 2200. The reinforcing portion is disposed at at least one segment of a reinforcing area. The reinforcing portion is constructed by forming at least one depression structure and/or at least one protrusion structure at the at least one segment. As shown in FIGS. 6 to 8, the reinforcing portion includes a groove 2300, and the groove 2300 extends from the needle sharp 2200 toward the base wall 2130 of the needle body 2100. The groove 2300 can be located at a first surface of the needle sharp 2200 facing toward the receiving space (not labeled in the second embodiment) and at a first surface of the base wall 2130 facing toward the receiving space, and the groove 2300 can be positioned at the central axis I1. A cross section of the groove is V-shaped or U-shaped. During the manufacture, the groove 2300 can be formed on the needle sharp portion (not shown in the second embodiment) and the base wall part (not shown in the second embodiment) of the flat blank (not shown in the second embodiment) first, and the depth of the groove 2300 is not larger than or equal to the thickness of the flat blank. The insertion needle structure 2000 having the groove 2300 can then be formed by bending the flat blank, and a second surface of the needle sharp 2200 facing away from the receiving space and a second surface of the base wall 2130 facing away from the receiving space are still smooth surfaces. Precisely, the groove 2300 is formed by pressing the first surface of the needle sharp portion and the first surface of the base wall portion. Please be noted that, during the manufacture, the groove 2300 extends no further than the part of the base wall portion adjacent to the needle sharp portion. The material density of the needle sharp 2200 will be increased after pressing, and the strength of the needle sharp 2200 can be enhanced, thereby favorable for increasing the anti-bending capability of the insertion needle structure 2000 and avoiding the needle sharp 2200 from bending or deforming during the implanting process. In other embodiments, the groove can also be formed by cutting off a part of the material. The reinforcing portion can include a plurality of grooves, the reinforcing portion can only be located on the needle sharp, and the present disclosure is not limited thereto.

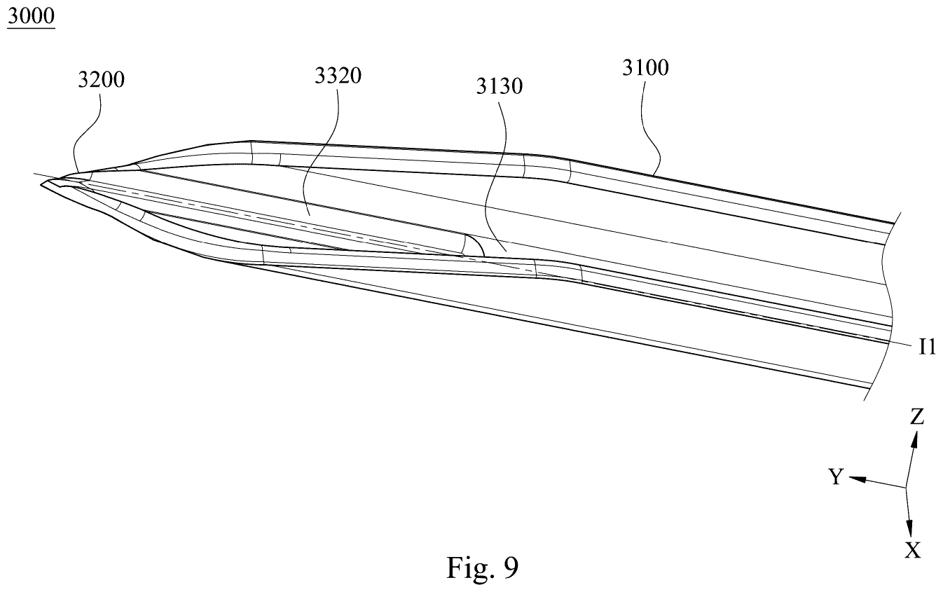

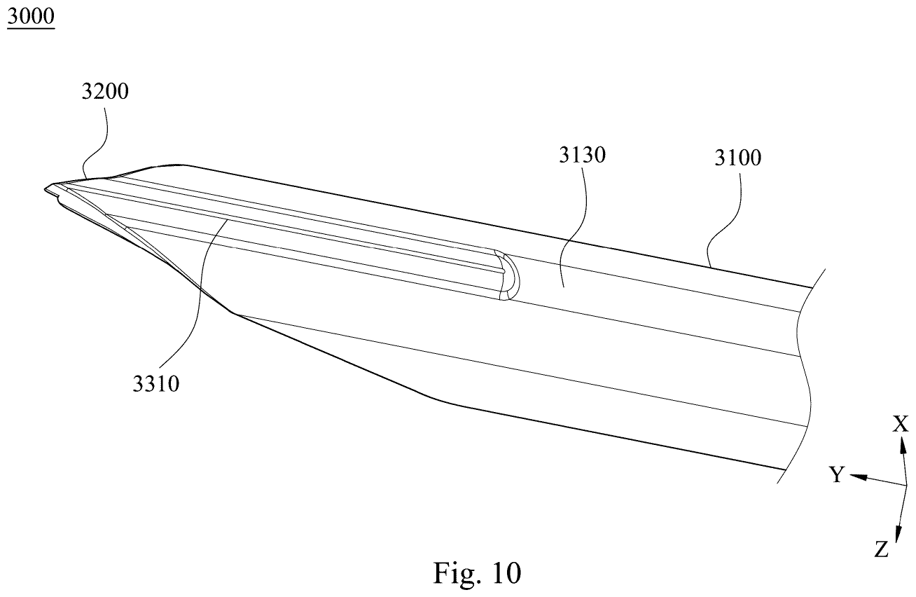

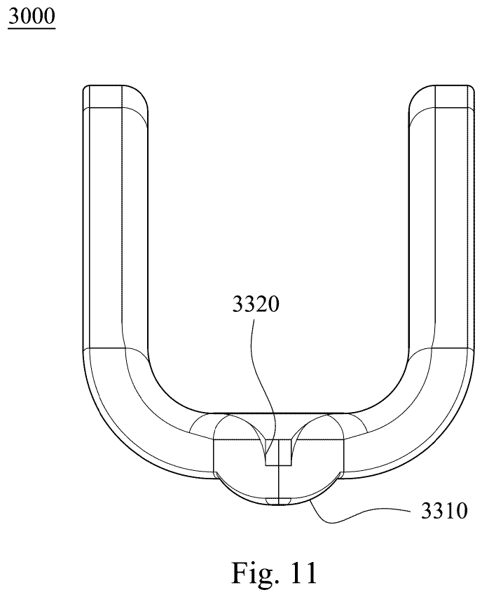

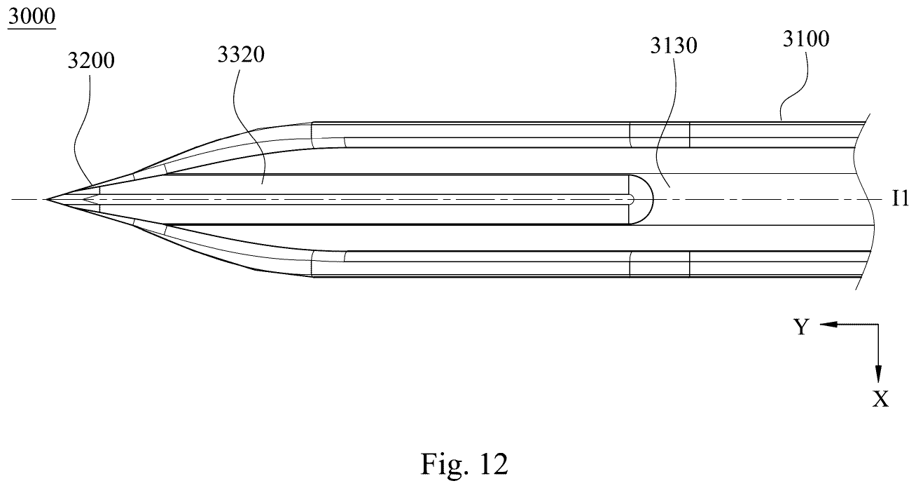

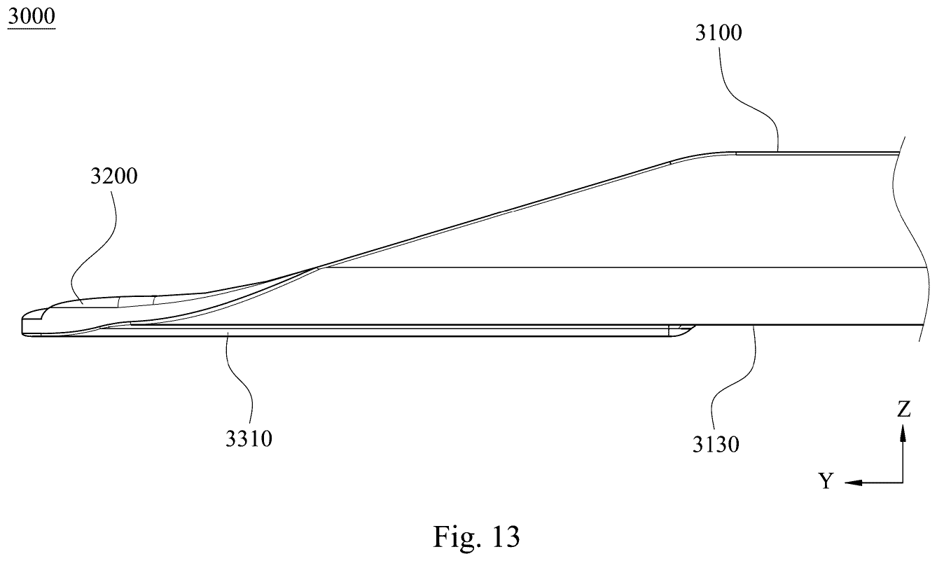

FIG. 9 shows one three-dimensional schematic view of an insertion needle structure 3000 according to a third embodiment of the present disclosure. FIG. 10 shows another three-dimensional schematic view of the insertion needle structure 3000 of the third embodiment of FIG. 9. FIG. 11 shows a front view of the insertion needle structure 3000 of the third embodiment of FIG. 9. FIG. 12 shows a top view of the insertion needle structure 3000 of the third embodiment of FIG. 9. FIG. 13 shows a side view of the insertion needle structure 3000 of the third embodiment of FIG. 9. The insertion needle structure 3000 is similar to the insertion needle structure 1000 of the first embodiment and includes a needle sharp 3200 and a needle body 3100. The difference is that the insertion needle structure 3000 can further include a reinforcing portion (not labeled). The reinforcing portion includes a rib 3310, and the rib 3310 extends from the needle sharp 3200 toward the base wall 3130 of the needle body 3100. To be more specific, the reinforcing portion can further include a pressed depression 3320, the pressed depression 3320 can be located at a first surface of the needle sharp 3200 facing toward the receiving space (not labeled in the third embodiment) and at a first surface of the base wall 3130 facing toward the receiving space, and the pressed depression 3320 can be positioned at the central axis I1. The rib 3310 is located at a second surface of the needle sharp 3200 facing away from the receiving space and at a second surface of the base wall 3130 facing away from the receiving space, and the rib 3310 can be positioned at the central axis I1. In other words, the pressed depression 3320 and the rib 3310 correspond to each other. During the manufacture, the pressed depression 3320 can be formed on the first surfaces of the needle sharp portion (not shown in the third embodiment) and the base wall part (not shown in the third embodiment) of the flat blank (not shown in the third embodiment) first, and the depth of the pressed depression 3320 is larger than the thickness of the flat blank, thereby automatically forming the rib 3310 protruding from the second surface. The insertion needle structure 3000 having the pressed depression 3320 and the rib 3310 can then be formed by bending the flat blank. Please be noted that, during the manufacture, the pressed depression 3320 extends no further than the part of the base wall portion adjacent to the needle sharp portion, and the rib 3310 extends no further than the part of the base wall portion adjacent to the needle sharp portion correspondingly. In the third embodiment, the pressed depression 3320 is pressed by the same pressing method, and the protruding rib 3310 which is thinner and has higher material density can be formed. Hence, the material density of the needle sharp 3200 will be increased after pressing, and the strength of the needle sharp 3200 can be enhanced, thereby favorable for increasing the anti-bending capability of the insertion needle structure 3000. The manufacturing process of the present disclosure is not limited thereto. Moreover, the reinforcing portion is not limited to the groove, e.g., the groove 2300 in the second embodiment, or the rib, e.g., the rib 3310 in the third embodiment, the reinforcing portion can be a projection protruding from the second surface of the needle sharp facing away from the receiving space, and the reinforcing portion can be only disposed at the needle sharp.

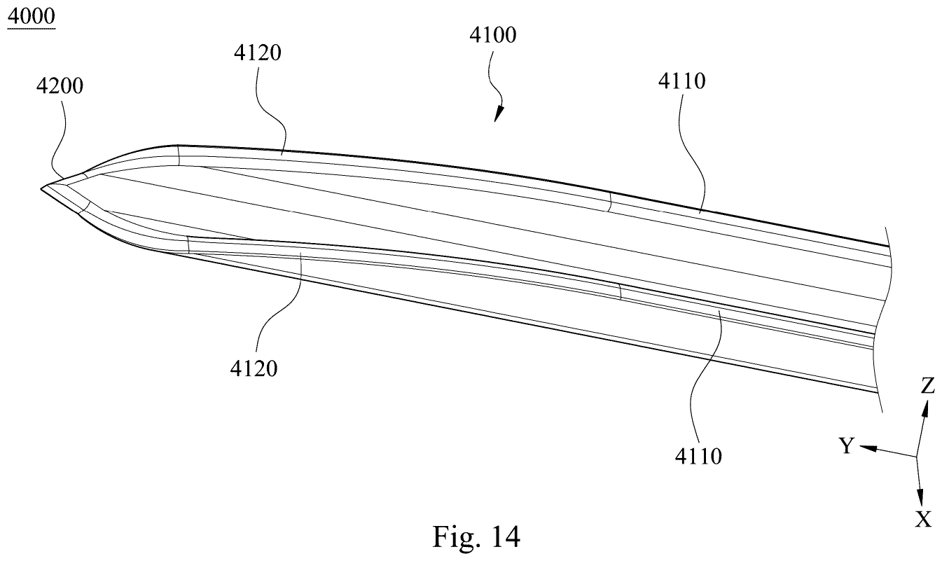

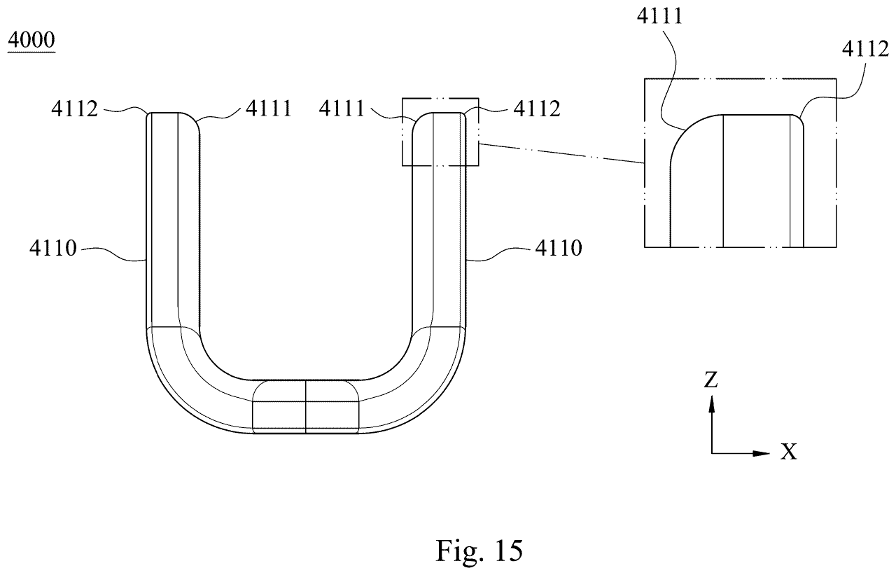

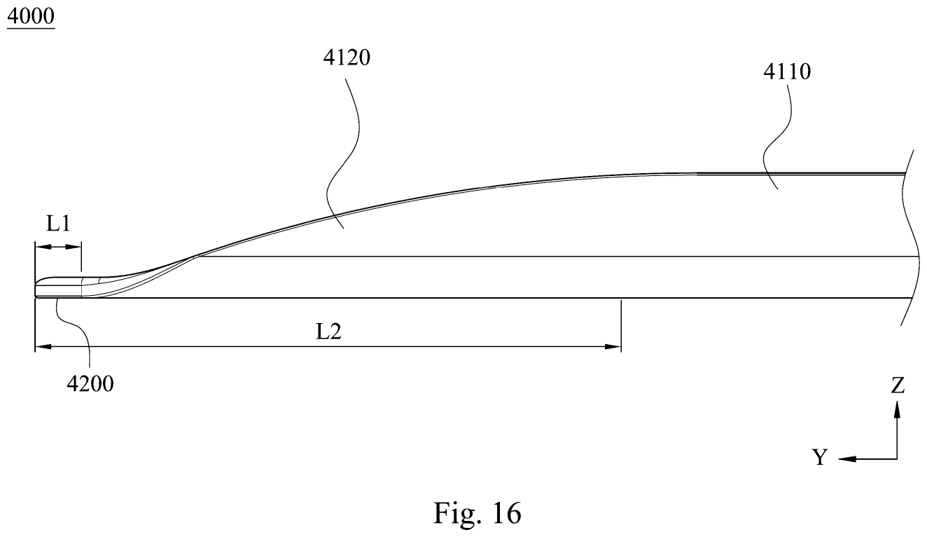

FIG. 14 shows a three-dimensional schematic view of an insertion needle structure 4000 according to a fourth embodiment of the present disclosure. FIG. 15 shows a front view of the insertion needle structure 4000 of the fourth embodiment of FIG. 14. FIG. 16 shows a side view of the insertion needle structure 4000 of the fourth embodiment of FIG. 14. The insertion needle structure 4000 is similar to the insertion needle structure 1000 and includes a needle sharp 4200 and a needle body 4100. The needle body 4100 includes two slope sections 4120 and two side walls 4110, and each of the side walls 4110 includes a first inner edge 4111 and a first outer edge 4112. The difference is that each of the slope sections 4120 is curved. In other words, the front end of the needle body 4100 of the insertion needle structure 4000 in the fourth embodiment connected to the needle sharp 4200 is curved. More particularly, a projected line generated by projecting the height of each of the slope sections 4120 onto the plane formed by the height direction Z and the length direction Y is a curved line with its convex vertex facing upward. The tangent slopes of different height positions of the slope section 4120 are different.

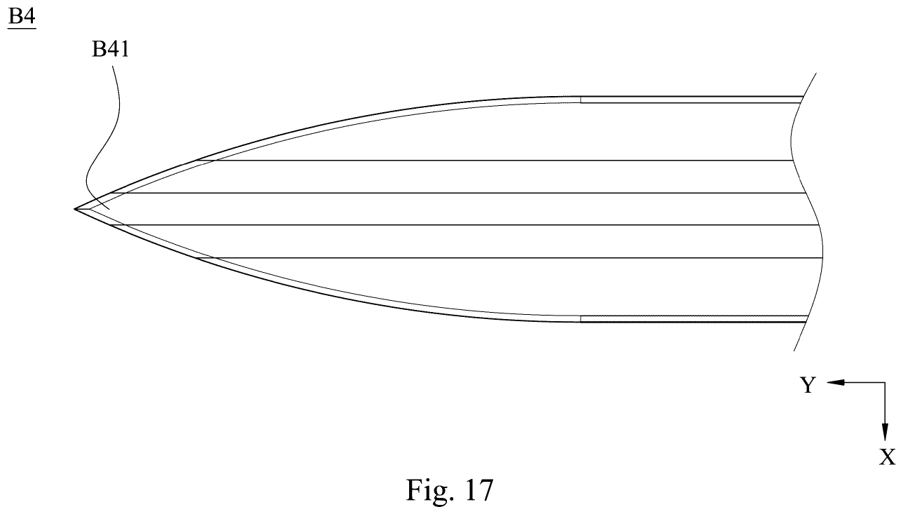

FIG. 17 shows a top view of a flat blank B4 used for being bended and forming the insertion needle structure 4000 of the fourth embodiment of FIG. 14. Please refer to FIG. 17 with reference to FIGS. 14 to 16, the flat blank B4 is used for being bended and forming the insertion needle structure 4000. The flat blank B4 is similar to the flat blank B1 of the first embodiment. The difference is that the inclined line extending from the needle sharp portion B41 is curved, and no obvious inflection point is presented at the intersection between the inclined line and the straight line. Therefore, the needle sharp 4200 of the insertion needle structure 4000 is shortened while the width thereof is increased, thereby increasing the structural strength of the needle sharp 4200 to avoid bending of the needle sharp 4200. Moreover, through the curved slope sections 4120, the aperture formed by piercing the skin surface of the organism can be smoothly expanded, and the smoothness of the implanting process can be increased. As a result, the piercing pain can be lowered, which facilitates for implanting the biosensor. In other embodiments, the curve of each of the slope sections can be increased, but the sharpness should be taken into consideration while modifying the curve of each of the slope sections.

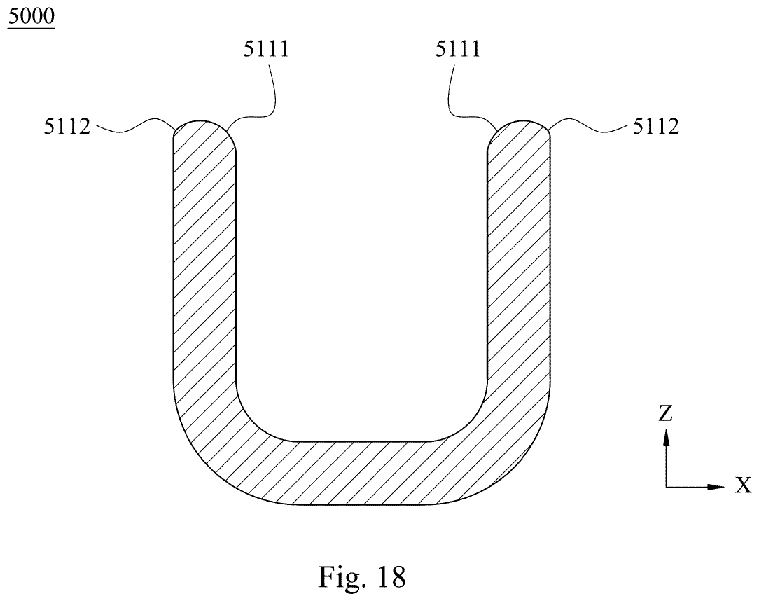

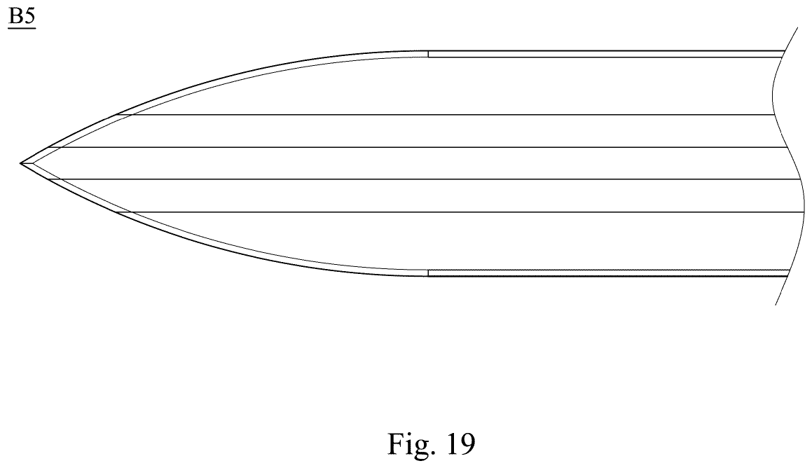

FIG. 18 shows a front section view of an insertion needle structure 5000 according to a fifth embodiment of the present disclosure. FIG. 19 shows a top view of a flat blank B5 used for being bended and forming the insertion needle structure 5000 of the fifth embodiment of FIG. 18. The insertion needle structure 5000 is similar to the insertion needle structure 1000 and includes two first inner edges 5111 and two first outer edges 5112, and each of the first inner edges 5111 is directly connected to each of the first outer edges 5112. In other words, the connecting surface 1150 of the first embodiment is omitted, and each of the first inner edges 5111 is allowed to be directly connected to each of the first outer edges 5112. In addition, the curve formed by the inclined line extending from the needle sharp portion (not labeled) of the flat blank B5 is larger than that of the flat blank B4 in the second embodiment, and the curve of the slope section can be increased.

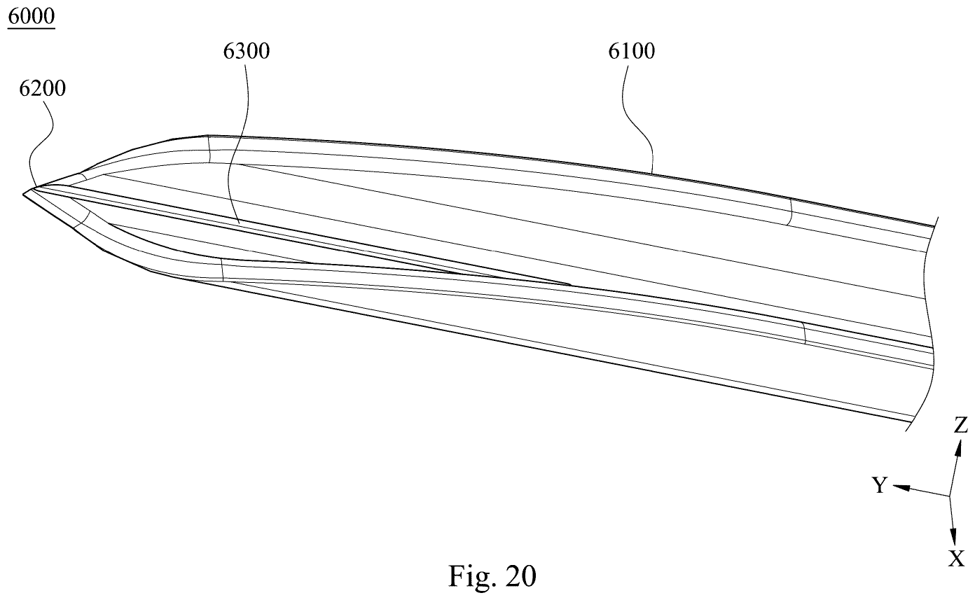

FIG. 20 shows a three-dimensional schematic view of an insertion needle structure 6000 according to a sixth embodiment of the present disclosure. The insertion needle structure 6000 is similar to the insertion needle structure 4000 of the fourth embodiment and includes a needle sharp 6200 and a needle body 6100. The difference is that the insertion needle structure 6000 can further include a reinforcing portion (not labeled). The reinforcing portion can include a groove 6300 whose manufacture process and structure are identical to that of the groove 2300 of the second embodiment, and the details will not be mentioned.

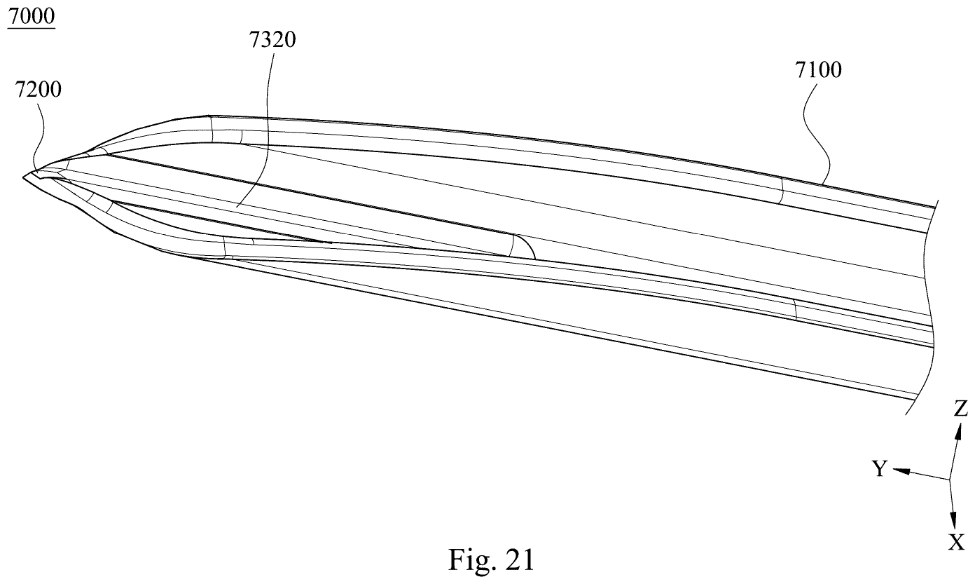

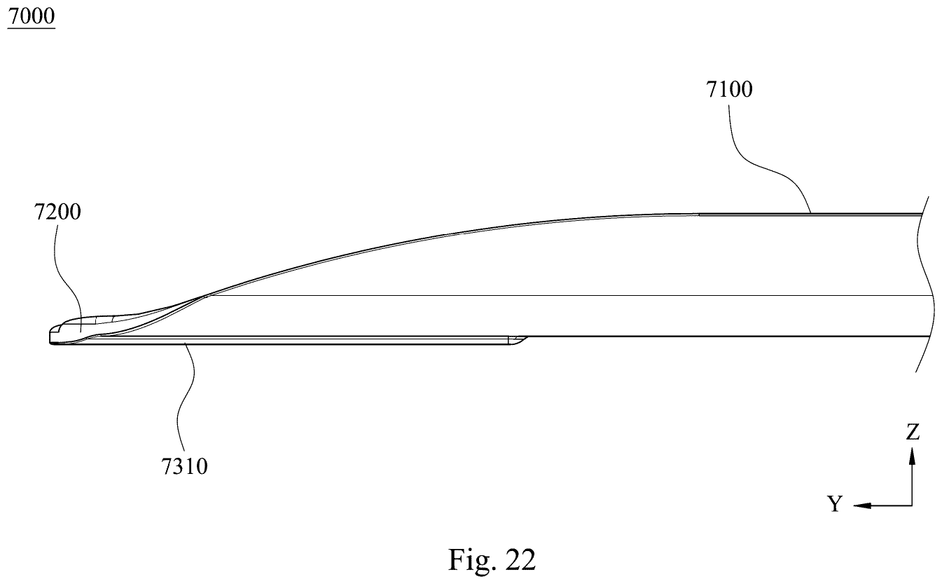

FIG. 21 shows a three-dimensional schematic view of an insertion needle structure 7000 according to a seventh embodiment of the present disclosure. FIG. 22 shows a side view of the insertion needle structure 7000 of the seventh embodiment of FIG. 21. The insertion needle structure 7000 is similar to the insertion needle structure 4000 of the fourth embodiment and includes a needle sharp 7200 and a needle body 7100. The difference is that the insertion needle structure 7000 can further include a reinforcing portion (not labeled). The reinforcing portion can include a rib 7310 and a pressed depression 7320 whose manufacture processes and structures are identical to that of the rib 3310 and the pressed depression 3320 of the third embodiment, and the details will not be mentioned.

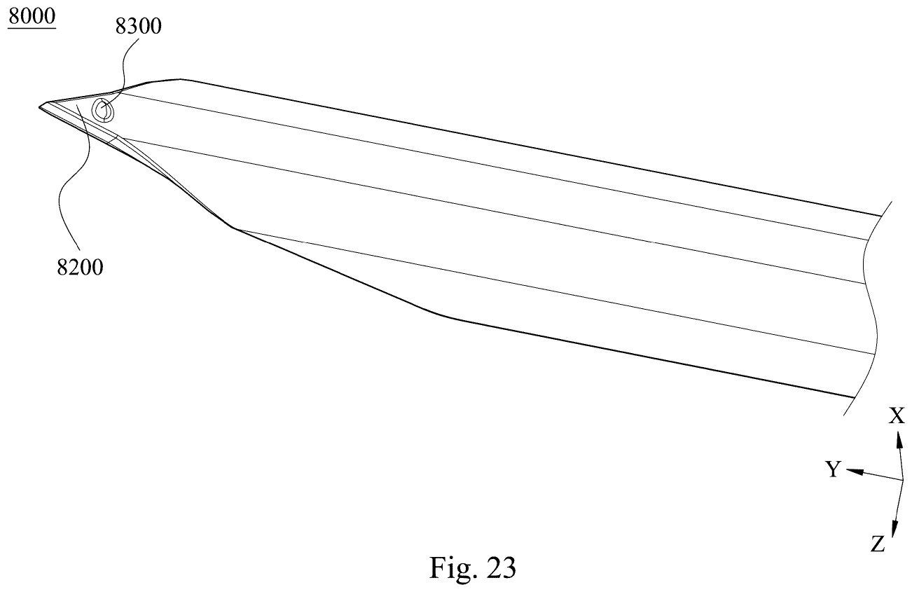

FIG. 23 shows a three-dimensional schematic view of an insertion needle structure 8000 according to an eighth embodiment of the present disclosure. The insertion needle structure 8000 is similar to the insertion needle structure 3000 of the third embodiment. The difference is that the reinforcing portion is a projection 8300 protruding from the second surface of the needle sharp 8200 facing away from the receiving space, and the projection 8300 is only located on the needle sharp 8200. The manufacture process and structure of the projection 8300 are identical to that of the rib 3310 and the pressed depression 3320 of the third embodiment, and the details will not be mentioned.

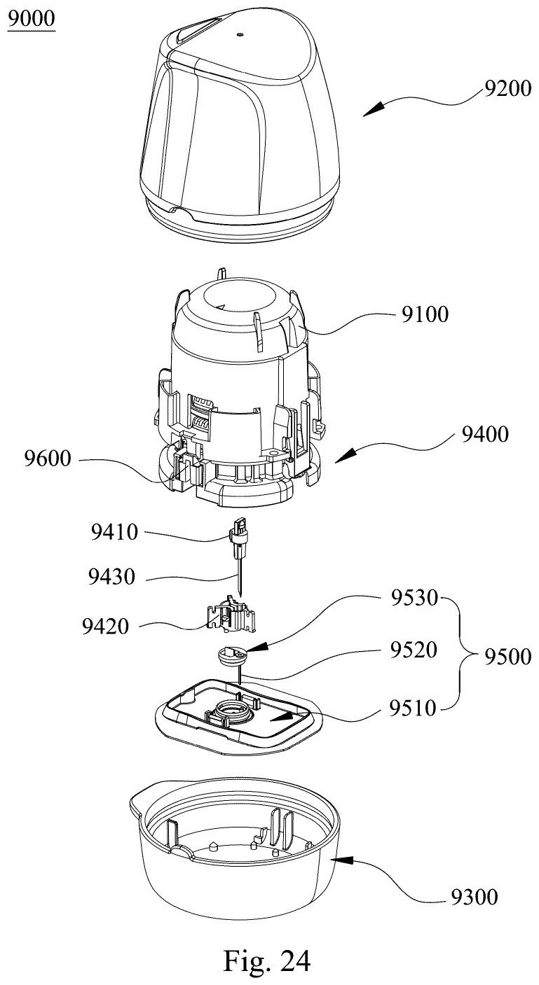

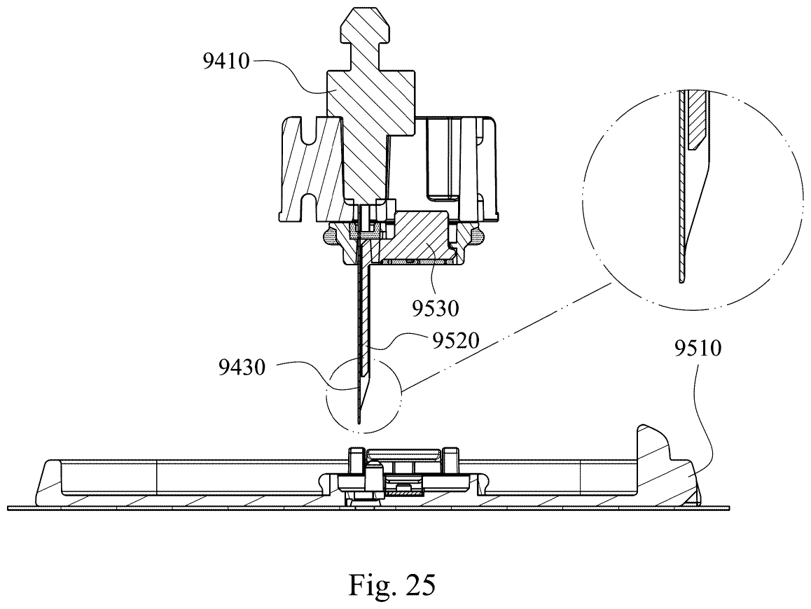

FIG. 24 shows an exploded three-dimensional schematic view of an inserter 9000 according to a ninth embodiment of the present disclosure. FIG. 25 shows a partial section view of the inserter 9000 of the ninth embodiment of FIG. 24. The inserter 9000 includes a cover 9100, an inserting module 9400 and a removing module 9500.

The cover 9100 has a main space (not labeled). The inserting module 9400 is disposed within the main space of the cover 9100 and includes an insertion needle structure 9430. The removing module 9500 can include a base 9510 and a biosensor 9520. The base 9510 is detachably limited within the inserting module 9400. The biosensor 9520 is detachably assembled with the base 9510 and at least a part thereof is received in the receiving space (not shown in the ninth embodiment) of the insertion needle structure 9430. When the cover 9100 is pressed downward, the inserting module 9400 is driven to allow the insertion needle structure 9430 to move downward so as to carry the biosensor 9520 to be implanted underneath a skin surface of an organism for conducting a measurement of a physiological signal inside the organism.

The inserter 9000 can further include an upper cap 9200, a lower cap 9300 and two fixing member 9600. A sealing space for receiving the cover 9100, the inserting module 9400 and the removing module 9500 is formed after the upper cap 9200 is engaged with the lower cap 9300. The two fixing member 9600 is symmetrically inserted into the inserting module 9400 to be detachably coupled to the base 9510. Each of the fixing members 9600 can include a supporting portion (not shown) for supporting a biosensor bracket 9530, and the biosensor bracket 9530 is configured to carry the biosensor 9520. The inserting module 9400 can further include an insertion needle member 9410 and an insertion needle supporting socket 9420. The insertion needle member 9410 is inserted into the insertion needle supporting socket 9420, and the insertion needle structure 9430 can be assembled with the insertion needle member 9410. The insertion needle structure 9430 can be any one of the insertion needle structures 1000, 2000, 3000, 4000, 5000, 6000, 7000 and 8000, and the present disclosure is not limited thereto.

During the operation, the user can press the upper cap 9200 downward to allow the cover 9100 inside the upper cap 9200 to move downward, which causes the fixing member 9600 to horizontally move so as to release the restriction between the fixing member 9600, the biosensor bracket 9530 and the base 9510. Moreover, through release of the prepressing elasticity of a first elastic member (not shown) inside the inserting module 9400, the insertion needle member 9410, the insertion needle structure 9430 and the biosensor 9520 can be implanted underneath the skin surface of the organism. Meanwhile, the biosensor bracket 9530 is assembled with the base 9510, and the biosensor 9520 is remained under the skin surface of the organism. After release of the prepressing elasticity of a second elastic member (not shown) inside the inserting module 9400, the insertion needle member 9410 can be retraced, thereby completing automatically implanting and retracing the insertion needle member 9410.

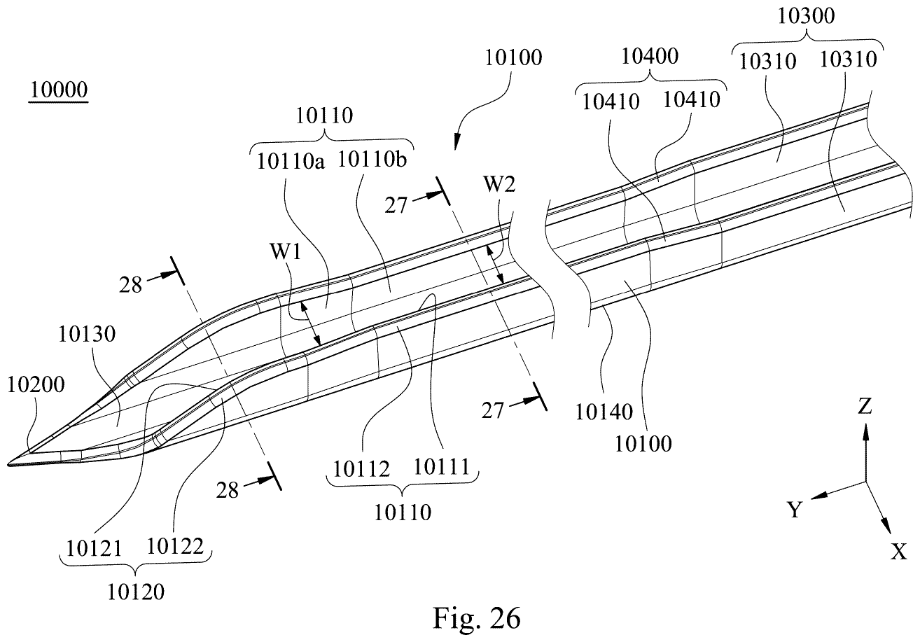

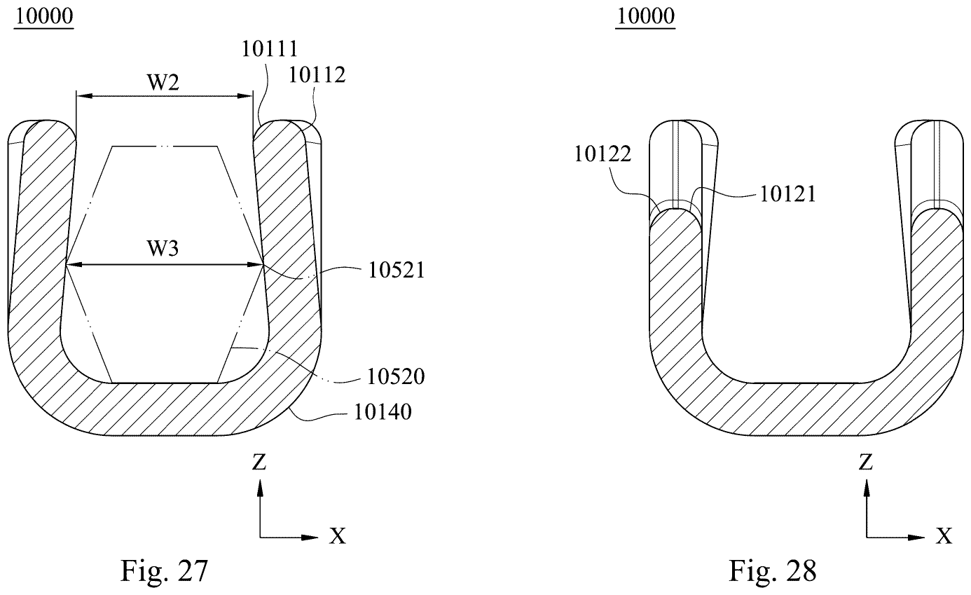

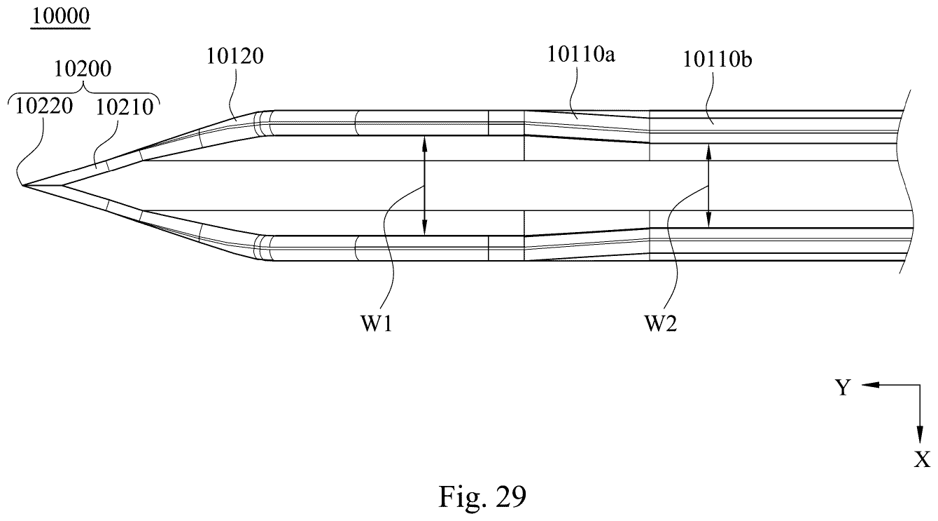

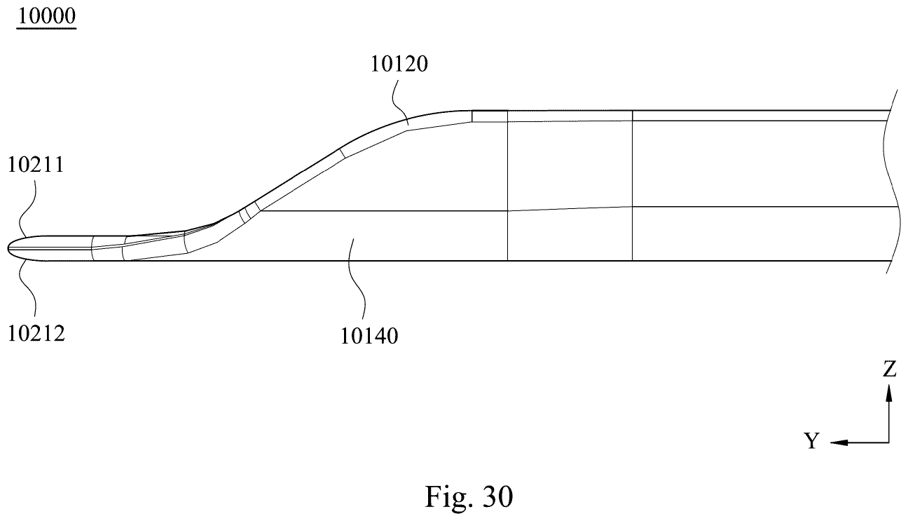

FIG. 26 shows a three-dimensional schematic view of an insertion needle structure 10000 according to a tenth embodiment of the present disclosure. FIG. 27 shows a cross-sectional view of the insertion needle structure 10000 of the tenth embodiment of FIG. 26 taken along line 27-27. FIG. 28 shows a cross-sectional view of the insertion needle structure 10000 of the tenth embodiment of FIG. 26 taken along line 28-28. FIG. 29 shows a top view of the insertion needle structure 10000 of the tenth embodiment of FIG. 26. FIG. 30 shows a side view of the insertion needle structure 10000 of the tenth embodiment of FIG. 26. The insertion needle structure 10000 is similar to the insertion needle structure 1000 of the first embodiment and includes a needle sharp 10200 and a needle body 10100. The needle body 10100 is integrally connected to the needle sharp 10200 and includes a base wall 10130, two side walls 10110, two slope sections 10120, and two curved connecting sections 10140. The needle body 10100 is for receiving a biosensor 10520.

Each of the side walls 10110 extends upward from each of the curved connecting sections 10140. Each of the side walls 10110 has a first section 10110a and a second section 10110b, and each of the first sections 10110a is connected to each of the slope sections 10120. Each of the second sections 10110b includes a flat surface, and the two flat surfaces are nonparallel.

Precisely, each of the side walls 10110 is divided into two sections, i.e., the first section 10110a and the second section 10110b, and each of the first sections 10110a is close to the slope sections 10120. Each of the first sections 10110a is not bended inward, and therefore two flat surfaces of the two first sections 10110a are parallel. Each of the second sections 10110b is bended inward from each of the curved connecting sections 10140, and the flat surfaces of the two second sections 10110b are not parallel. A wall aperture is formed between the two first sections 10110a and has a width W1 along the width direction X. Another wall aperture is formed between the two second sections 10110b and has a width W2 along the width direction X. The width W1 is larger than the width W2.

The insertion needle structure 10000 may further include an assembling body 10300 and a connecting body 10400. The assembling body 10300 is connected to an insertion needle member of an inserter, the connecting body 10400 is connected between the assembling body 10300 and the needle body 10100. The assembling body 10300 has two flat walls 10310 parallel to each other, the connecting body 10400 has two connecting walls 10410, each of the two connecting walls 10410 is connected between each of the flat walls 10310 and each of the side walls 10110, and each of the two connecting walls 10410 is partially inclined from each of the flat walls 10310 toward each of the side walls 10110. A width of a wall aperture between the flat walls 10310 along the width direction X is equal to the width W1, and therefore the two connecting walls 10410 are partially inclined toward the side walls 10110.

As shown in FIG. 27, the biosensor 10520 has a widest portion 10521 having a maximum width W3 along the width direction X of the insertion needle structure 10000, the width W2 is smaller than the maximum width W3 of the biosensor 10520. Therefore, the biosensor 10520 may not separate from the insertion needle structure 10000 in the height direction Z. It should be noted that FIG. 27 is only used to illustrate that the biosensor 10520 has the maximum width W3; however, a gap between the biosensor 10520 and each of the side walls 10110 is necessary. Additionally, a difference between the maximum width W3 of the biosensor 10520 and the width W1 of the wall aperture, as well as a difference between the maximum width W3 of the biosensor 10520 and the width W2 of the wall aperture, is in the range of-100 um to 100 um.

Each of the side walls 10110 may have a first inner edge 10111 and a first outer edge 10112, and each of the first inner edges 10111 is curved. Moreover, each of the first outer edges 10112 is curved. R11 represents a radius of each of the first inner edges 10111, R12 represents a radius of each of the first outer edges 10112, and a condition of R11≥R12 is satisfied. In the tenth embodiment, each of the first inner edges 10111 is directly connected to each of the first outer edges 10112, that is, no connecting surface is connected between the first inner edge 10111 and the first outer edge 10112. However, in other embodiments, a connecting surface may be connected between the first inner edge and the first outer edge.

As shown in FIGS. 26 to 30, each of the slope sections 10120 has a second inner edge 10121 connected to the first inner edge 10111, and a second outer edge 10122 connected to the first outer edge 10112. Each of the second inner edges 10121 is curved, and each of the second outer edges 10122 is curved. The needle sharp 10200 includes two slants 10210 connected to the two curved connecting sections 10140, respectively, and the two slants 10210 intersect at a needle tip 10220. Each of the slants 10210 includes a needle sharp top edge 10211 being curved and connected to each of the two second inner edges 10121, and a needle sharp bottom edge 10212 being curved and connected to each of the second outer edges 10122.

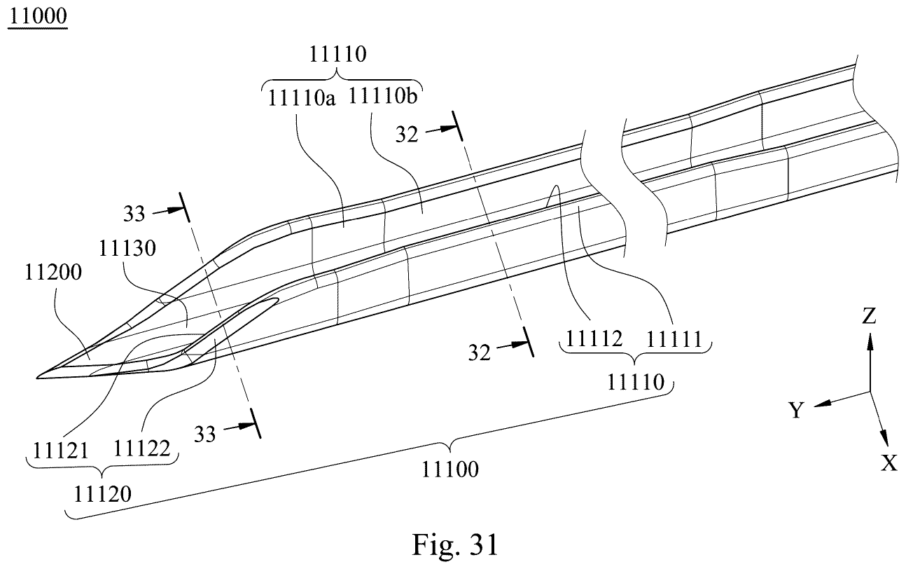

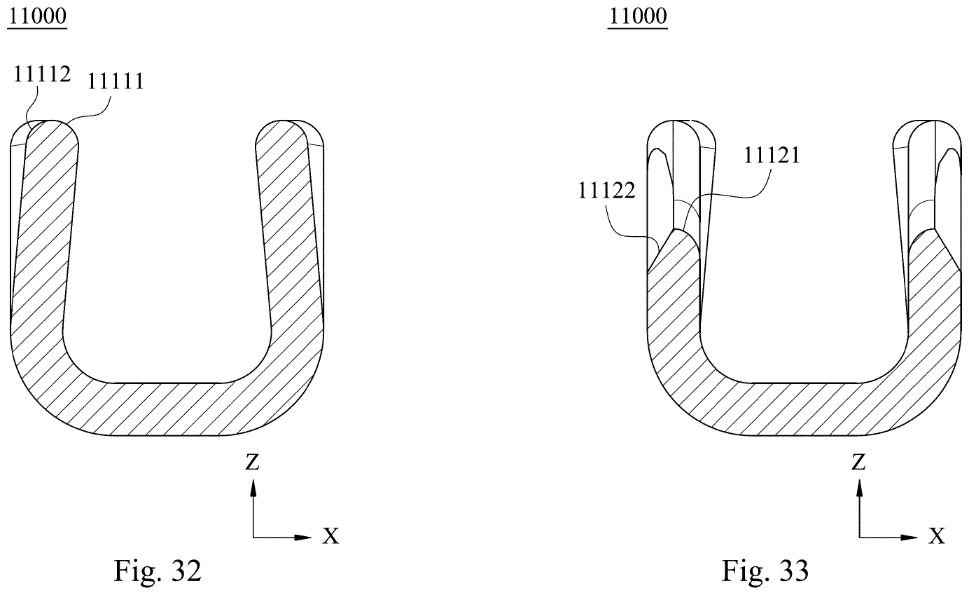

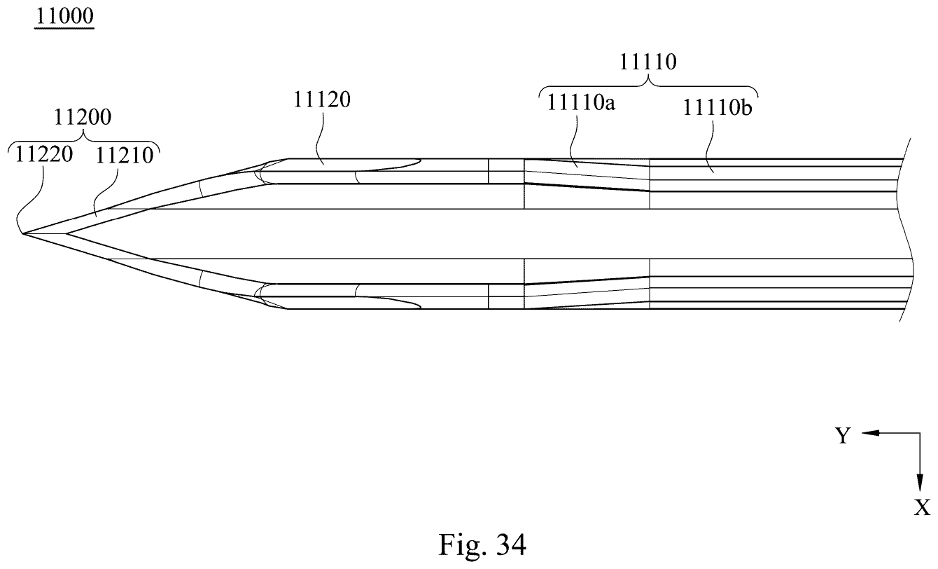

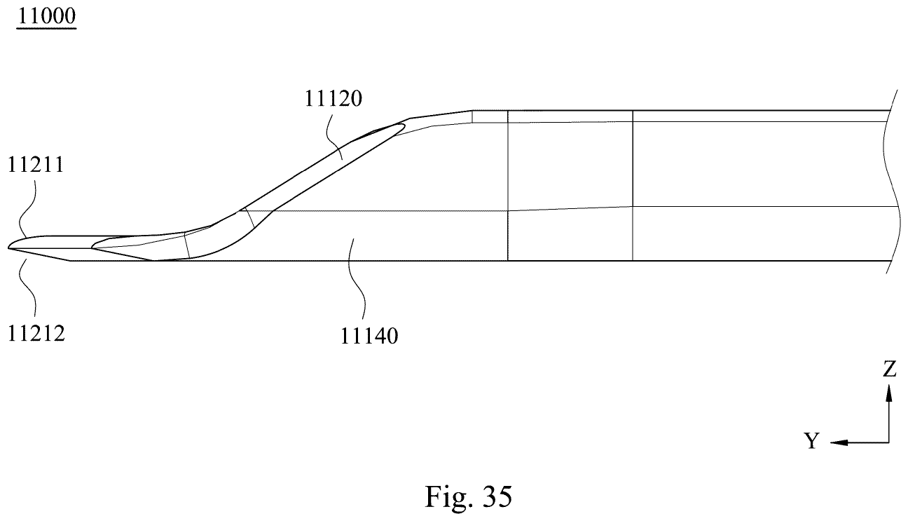

FIG. 31 shows a three-dimensional schematic view of an insertion needle structure 11000 according to an eleventh embodiment of the present disclosure. FIG. 32 shows a cross-sectional view of the insertion needle structure 11000 of the eleventh embodiment of FIG. 31 taken along line 32-32. FIG. 33 shows a cross-sectional view of the insertion needle structure 11000 of the eleventh embodiment of FIG. 31 taken along line 33-33. FIG. 34 shows a top view of the insertion needle structure 11000 of the eleventh embodiment of FIG. 31. FIG. 35 shows a side view of the insertion needle structure 11000 of the eleventh embodiment of FIG. 31. The insertion needle structure 11000 is similar to the insertion needle structure 10000 of the tenth embodiment and includes a needle sharp 11200 and a needle body 11100. The needle body 11100 is integrally connected to the needle sharp 11200 and includes a base wall 11130, two side walls 11110, two slope sections 11120, and two curved connecting sections 11140.

Each of the side walls 11110 includes a first section 11110a and a second section 11110b, the structure of the side walls 11110 is similar to the structure of the side walls 10110 of the tenth embodiment, and a first inner edge 11111 and a first outer edge 11112 of each of the side walls 11110 are curved.

Each of the slope sections 11120 has a second inner edge 11121 connected to the first inner edge 11111, and a second outer edge 11122 connected to the first outer edge 11112. Each of the second inner edges 11121 is curved, and each of the second outer edges 11122 is partially flat and inclined in a direction away from the receiving space. In other words, a part of each of the slope sections 11120 is cut to be flat, and another part of each of the slope sections 11120 near each of the side walls 11110 remains being curved.

The needle sharp 11200 includes two slants 11210 connected to the two curved connecting sections 11140, respectively, and the two slants 11210 intersect at a needle tip 11220. A needle sharp top edge 11211 of each of the slants 11210 is curved, and a needle sharp bottom edge 11212 is flat to connect to the flat second outer edge 11122 of each of the slope sections 11120.

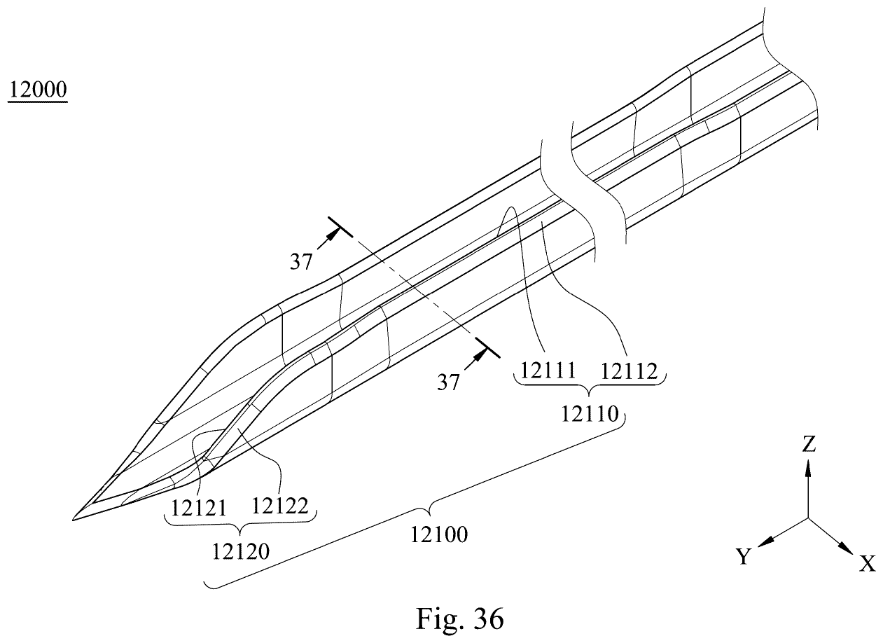

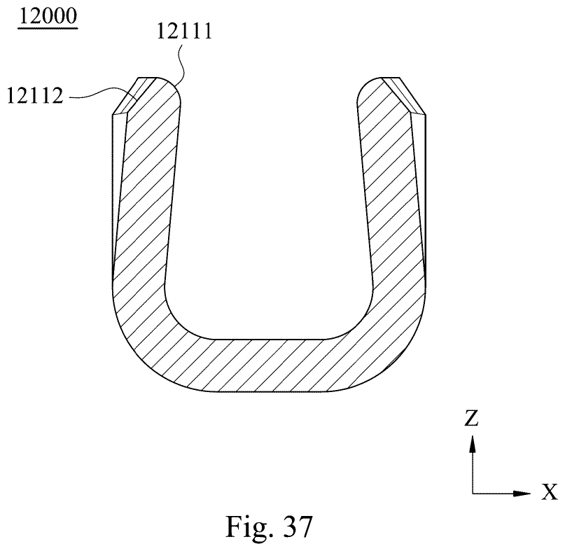

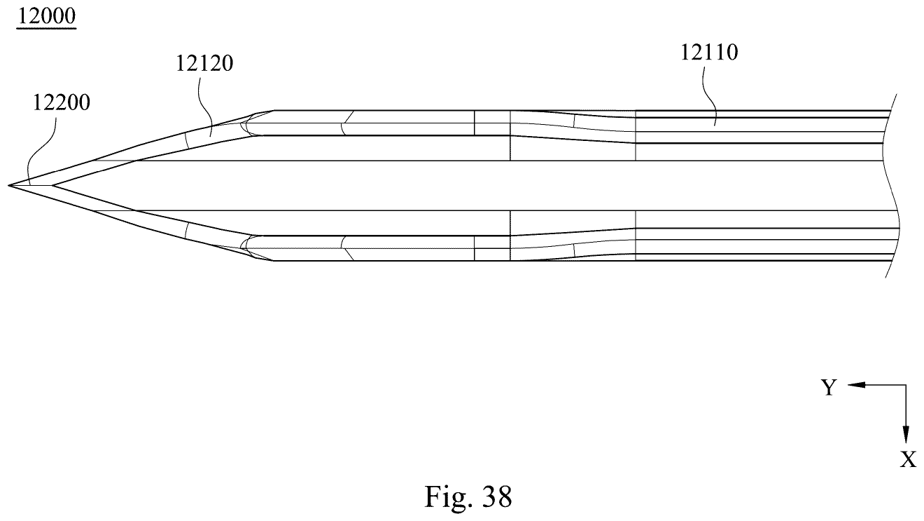

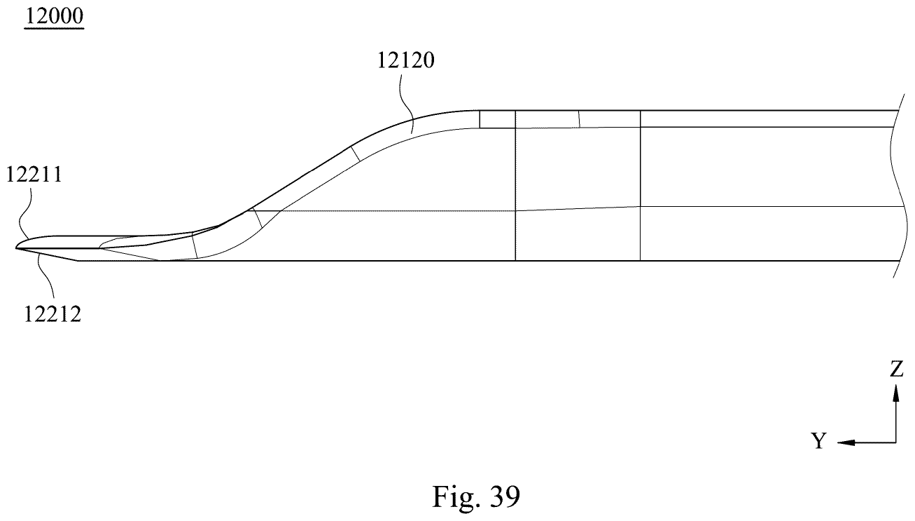

FIG. 36 shows a three-dimensional schematic view of an insertion needle structure 12000 according to a twelfth embodiment of the present disclosure. FIG. 37 shows a cross-sectional view of the insertion needle structure 12000 of the twelfth embodiment of FIG. 36 taken along line 37-37. FIG. 38 shows a top view of the insertion needle structure 12000 of the twelfth embodiment of FIG. 36. FIG. 39 shows a side view of the insertion needle structure 12000 of the twelfth embodiment of FIG. 36. The insertion needle structure 12000 is similar to the insertion needle structure 11000 of the eleventh embodiment and includes a needle sharp 12200 and a needle body 12100. The needle body 12100 includes two side walls 12110 and two slope sections 12120.

A first inner edge 12111 of each of the side walls 12110 is curved, and a first outer edge 12112 of each of the side walls 12110 is flat and inclined in a direction away from the receiving space. A second inner edge 12121 of each of the slope sections 12120 is curved, and a second outer edge 12122 of each of the slope sections 12120 is flat and inclined in a direction away from the receiving space. A needle sharp top edge 12211 is curved, and a needle sharp bottom edge 12212 is flat and inclined in a direction away from the receiving space.

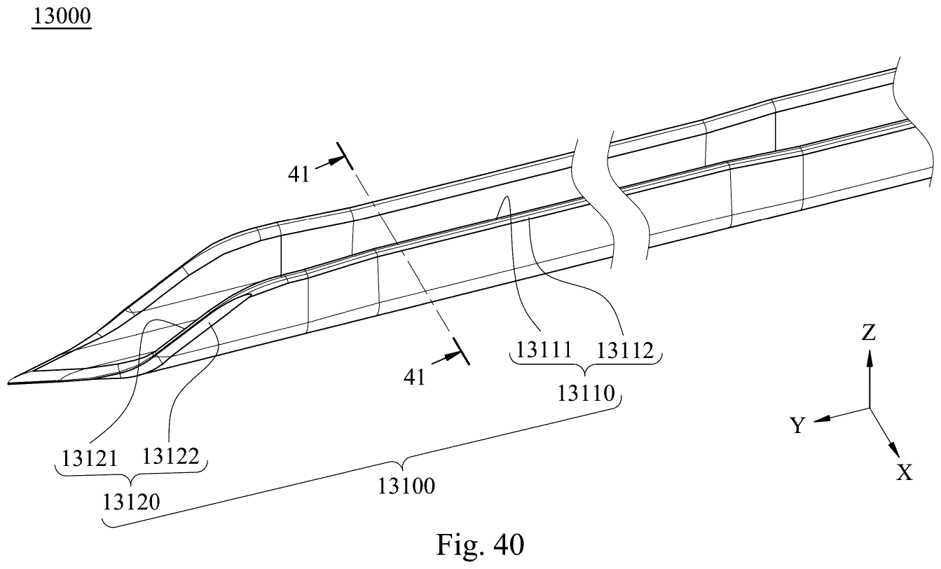

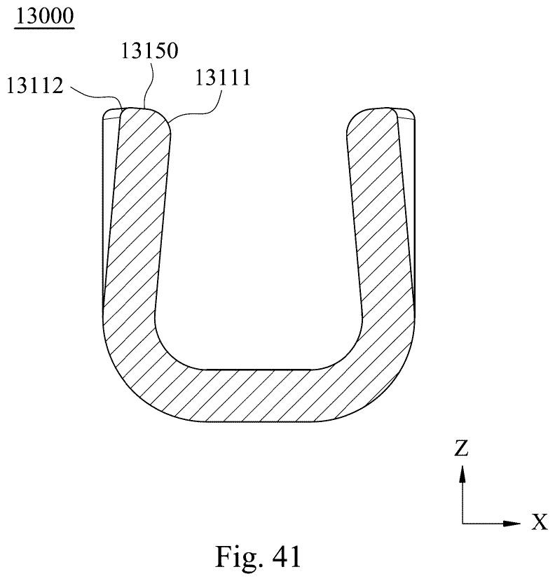

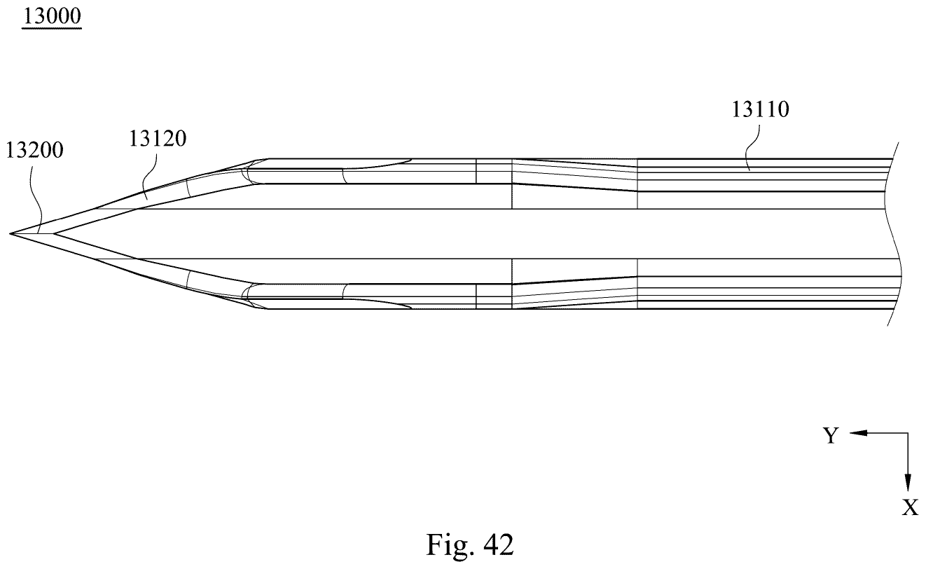

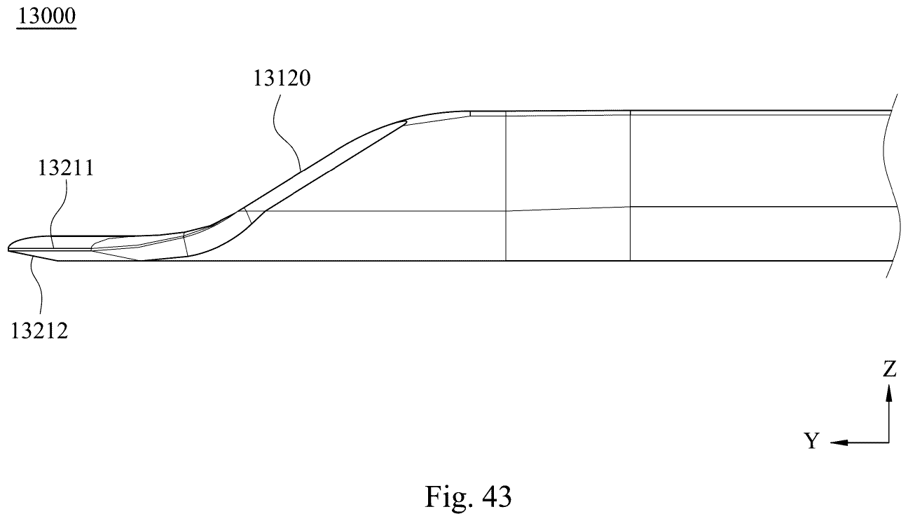

FIG. 40 shows a three-dimensional schematic view of an insertion needle structure 13000 according to a thirteenth embodiment of the present disclosure. FIG. 41 shows a cross-sectional view of the insertion needle structure 13000 of the thirteenth embodiment of FIG. 40 taken along line 41-41. FIG. 42 shows a top view of the insertion needle structure 13000 of the thirteenth embodiment of FIG. 40. FIG. 43 shows a side view of the insertion needle structure 13000 of the thirteenth embodiment of FIG. 40. The insertion needle structure 13000 is similar to the insertion needle structure 11000 of the eleventh embodiment and includes a needle sharp 13200 and a needle body 13100. The needle body 13100 includes two side walls 13110 and two slope sections 13120.

A first inner edge 13111 and a first outer edge 13112 of each of the side walls 13110 are curved, and a connecting surface 13150 is connected therebetween. A second inner edge 13121 of each of the slope sections 13120 is curved, and a second outer edge 13122 of each of the slope sections 13120 is partially flat and inclined in a direction away from the receiving space. A needle sharp top edge 13211 is curved, and a needle sharp bottom edge 13212 is flat and inclined in a direction away from the receiving space.

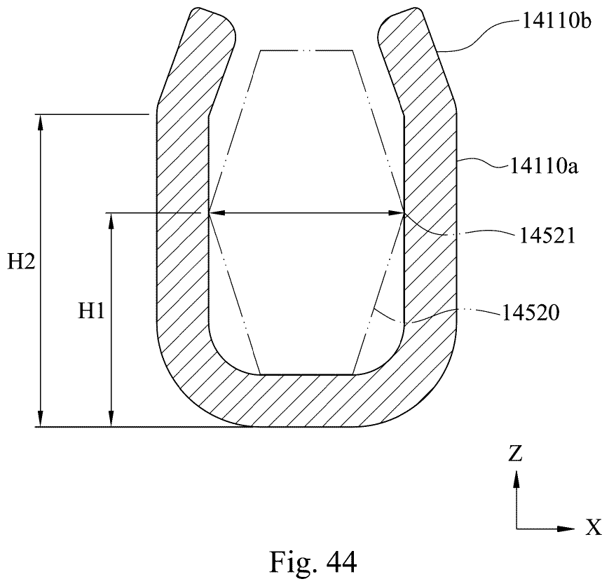

FIG. 44 shows a cross-sectional view of an insertion needle structure according to a fourteenth embodiment of the present disclosure, and it is noted that FIG. 44 is a cross-sectional surface. In the fourteenth embodiment, the second section of each of the side walls includes a straight portion 14110a and a bending portion 14110b. Each of the straight portions 14110a extends upward from each of the curved connecting sections, and each of the bending portions 14110b extends upward and inward from each of the straight portions 14110a. Each of the straight portions 14110a has a flat surface, and the two flat surfaces are parallel. Precisely, each of the second sections extends upward for at least a portion and then is bended inward to form the straight portion 14110a and the bending portion 14110b. A height between a bottom of the base wall and a widest portion 14521 of a biosensor 14520 along the height direction Z of the insertion needle structure is represented by H1. A height of the straight portion 14110a along the height direction Z is represented by H2, and a condition of H2>H1 is satisfied. Therefore, the straight portion 14110a is high enough to receive the biosensor 14520.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure covers modifications and variations of this disclosure provided they fall within the scope of the following claims.

What is claimed is:

1. An insertion needle structure, which is formed by bending a flat blank and is configured for receiving and allowing a biosensor to be partially implanted underneath a skin surface of an organism, the insertion needle structure comprising:
a needle sharp; and
a needle body integrally connected to the needle sharp and having a receiving space for receiving the biosensor, the needle body comprising:
a base wall;
two side walls located at two sides of the base wall, respectively, the two side walls being at least partially nonparallel, each of the two side walls being at least partially flat;
two slope sections located at the two sides of the base wall, respectively, each of the slope sections connected between each of the side walls and the needle sharp, each of the slope sections being curved; and
two curved connecting sections located at the two sides of the base wall, each of the curved connecting sections being connected between each of the side walls and the base wall and between each of the slope sections and the base wall;
wherein the needle sharp extends from the base wall and the curved connecting sections.

2. The insertion needle structure of claim 1, wherein each of the side walls has a first inner edge and a first outer edge, each of the first inner edges is near the receiving space, each of the first outer edges faces away from the receiving space, and each of the first inner edges is curved.

3. The insertion needle structure of claim 2, wherein each of the first outer edges is curved, R11 represents a radius of each of the first inner edges, R12 represents a radius of each of the first outer edges, and a condition of R11≥R12 is satisfied.

4. The insertion needle structure of claim 3, wherein the flat blank has a thickness represented by T1, and a condition of 20%≤R11/T1≤50% is satisfied.

5. The insertion needle structure of claim 2, wherein each of the slope sections has a second inner edge connected to the first inner edge, and a second outer edge connected to the first outer edge, and each of the second inner edges is curved.

6. The insertion needle structure of claim 5, wherein each of the second outer edges is partially flat and inclined in a direction away from the receiving space.

7. The insertion needle structure of claim 5, wherein the needle sharp comprises two slants connected to the two curved connecting sections, respectively, the two slants intersect at a needle tip, and each of the slants comprises:
a needle sharp top edge being curved and connected to each of the second inner edges; and
a needle sharp bottom edge being curved or flat and connected to each of the second outer edges.

8. The insertion needle structure of claim 7, wherein the needle sharp bottom edge is flat, and each of the second inner edges is partially flat.

9. The insertion needle structure of claim 1, wherein each of the side walls extends upward from each of the curved connecting sections, each of the side walls has a first section and a second section, each of the first sections is connected to each of the slope sections, each of the second sections comprises a flat surface, and the flat surfaces are nonparallel.

10. The insertion needle structure of claim 1, wherein each of the side walls has a first section and a second section, each of the first sections is connected to each of the slope sections, each of the second sections comprises a straight portion and a bending portion, each of the straight portions extends upward from each of the curved connecting sections, each of the bending portions extends upward and inward from each of the straight portions, each of the straight portions has a flat surface, and the flat surfaces are parallel.

11. The insertion needle structure of claim 10, wherein the biosensor has a widest portion having a maximum width along a width direction of the insertion needle structure, a height between a bottom of the base wall and the widest portion of the biosensor along a height direction of the insertion needle structure is represented by H1, a height of the straight portion along the height direction is represented by H2, and a condition of H2>H1 is satisfied.

12. The insertion needle structure of claim 10, wherein the biosensor has a widest portion having a maximum width along a width direction of the insertion needle structure, a wall aperture is formed between the second sections, and a width of the wall aperture between the second sections along the width direction is smaller than the maximum width of the biosensor.

13. The insertion needle structure of claim 10, wherein the first sections are parallel, a wall aperture between the first sections along a width direction is larger than a wall aperture between the second sections.

14. The insertion needle structure of claim 1, further comprising an assembling body and a connecting body, wherein the assembling body is connected to an insertion needle member of an inserter, the connecting body is connected between the assembling body and the needle body, the assembling body has two flat walls parallel to each other, the connecting body has two connecting walls, each of the two connecting walls is connected between each of the flat walls and each of the side walls, and each of the two connecting walls is partially inclined from each of the flat walls toward each of the side walls.

15. The insertion needle structure of claim 1, further comprising a reinforcing portion disposed at at least one segment of a reinforcing area, wherein the reinforcing area is defined as the needle sharp and a part of the needle body adjacent to the needle sharp, the reinforcing portion is constructed by forming at least one depression structure and/or at least one protrusion structure at the at least one segment to avoid the needle sharp from bending or deforming by a force during an implanting process.

16. The insertion needle structure of claim 15, wherein the reinforcing portion comprises a groove extending from the needle sharp toward the base wall of the needle body, and a cross section of the groove is V-shaped or U-shaped.

17. The insertion needle structure of claim 15, wherein the reinforcing portion comprises a rib extending from the needle sharp toward the base wall of the needle body.

18. The insertion needle structure of claim 1, wherein each of the curved connecting sections has a height thereof represented by T2 along a height direction of the insertion needle structure, the flat blank has a thickness represented by T1, and a condition of T2/T1≥1.5 is satisfied.

19. An inserter, comprising:
a cover having a main space;
an inserting module disposed within the main space of the cover and comprising an insertion needle structure of claim 1; and
a removing module comprising:
a base detachably limited within the inserting module; and
the biosensor detachably assembled with the base and at least a part thereof received in the receiving space of the insertion needle structure;
wherein when the cover is pressed downward, the inserting module is driven to allow the insertion needle structure to move downward so as to carry the biosensor to implant underneath the skin surface of the organism for conducting a measurement of a physiological signal inside the organism.
